(19) United States
(12) Patent Application Publication
(10) Pub. No.: US 2005/0060815 A1
(43) Pub. Date: Mar. 24, 2005

(54) Novel heteroaromatic trinuclear black direct dyes

(76) Inventors: Sylvain Kravtchenko, Asnieres (FR); Alain Lagrange, Coupvray (FR); Herve David, (US); Andrew Greaves, Montevrain (FR); Nicole Bonaventure, Vincennes (FR); Laurent Vidal, Paris (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/678,635

(22) Filed: Oct. 6, 2003

Related U.S. Application Data
(60) Provisional application No. 60/431,749, filed on Dec. 9, 2002.

(30) Foreign Application Priority Data
Oct. 4, 2002 (FR) 02 12385

(51) Int. Cl. A61K 7/13

(52) U.S. Cl. 8/405

(57) ABSTRACT

The invention relates to novel heteroaromatic trinuclear direct dyes, to dye compositions containing these dyes and to the process for dyeing keratin fibres using them. In particular, the invention relates to heteroaromatic trinuclear direct dyes comprising a pyridine nucleus. These novel dyes make it possible to obtain black shades that show good fastness and good stability in the dye compositions.

The invention relates to novel heteroaromatic trinuclear direct dyes, to dye compositions containing these dyes and to the process for dyeing keratin fibres using them. In particular, the invention relates to heteroaromatic trinuclear direct dyes comprising a pyridine nucleus.

It is known practice to dye keratin fibres, and in particular human hair, with dye compositions containing direct dyes. The standard dyes that are used are in particular dyes of the nitrobenzene, anthraquinone, nitropyridine, azo, cationic azo, xanthene, acridine, azine, and triarylmethane nitrobenzene type or natural dyes.

These dyes, which are coloured and colouring molecules that show affinity for fibres, are applied to keratin fibres for the time required to obtain the desired coloration, and then rinsed out.

The colorations resulting therefrom are particularly chromatic colorations, but are, however, temporary or semi-permanent since the nature of the interactions that link the direct dyes to the keratin fibre, and their desorption from the surface and/or from the core of the fibre are responsible for their weak dyeing power and their poor wash-fastness or resistance to perspiration. These direct dyes are also generally light-sensitive due to the poor resistance of the chromophore to photochemical attack, and lead to fading of the coloration of the hair over time.

Patent application EP 1 166 754 describes a dye composition comprising cationic phenazinium azo direct dyes. Due to the presence of the azo function, these compounds are unstable when placed in contact with a reducing agent such as erythorbic acid, metabisulphite or sulphite, this instability being reflected in destruction of the chromophoric system.

The aim of the present invention is to provide novel direct dyes that do not have the drawbacks of the prior art, in particular direct dyes that allow dark, black to grey, shades to be obtained, which are light-fast, and resistant to bad weather, washing and perspiration, and are also stable in a standard dyeing medium.

In particular, the aim of the present invention is to provide black direct dyes allowing hair to be dyed in shades ranging from grey to black, without the need to lighten the hair beforehand, and also black direct dyes which, even when fading, do not change shade, for example by changing colour after the action of washing, light or sweat towards shades with blue, violet, red, green, etc. glints. Finally, these black direct dyes should make it possible to maintain, after several applications, the shade obtained during the first application.

These aims are achieved with the present invention, one subject of which is a dye composition comprising, in a suitable medium, a compound of formula (I) below or an addition salt thereof: 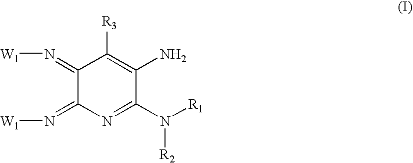
in which
R₃ represents:
   a hydrogen atom,
   a linear or branched C₁-C₁₀ hydrocarbon-based chain, which can form one or more 4- to 8-membered carbon-based rings, and which may be saturated or unsaturated, one or more carbon atoms of the carbon-based chain of which may be replaced with an oxygen, nitrogen or sulphur atom or with an SO₂ group; R₃ not comprising a peroxide bond or diazo or nitroso radicals,
   NR′₁R′₂, R′₁ and R′₂ being as defined for R₁ and R₂,
R₁ and R₂ represent, independently of each other:
   a hydrogen atom,
   a linear or branched C₁-C₁₀ hydrocarbon-based chain, which can form one or more 4- to 8-membered carbon-based rings, and which may be saturated or unsaturated, one or more carbon atoms of the carbon-based chain of which may be replaced with an oxygen, nitrogen or sulphur atom or with an SO₂ group; R₁ and R₂ not comprising a peroxide bond or diazo or nitroso radicals, and R₁ and R₂ not being directly linked to the nitrogen atom via an oxygen, sulphur or nitrogen atom or SO₂,
   an onium radical Z, or
R₁ and R₂ form, together with the nitrogen atom to which they are attached, a ring of formula (II): 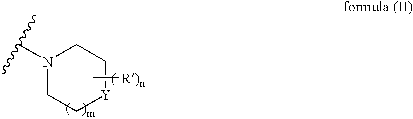
   in which
R′ represents:
   a hydrogen atom;
   a halogen atom such as fluorine, chlorine or bromine;
   a C₁-C₄ alkyl radical optionally substituted with one or more radicals chosen from hydroxyl, carboxyl, C₁-C₄ alkoxycarbonyl, (C₁-C₄)alkylamido ((C₁-C₄)alkylCONH—), (C₁-C₄)alkylcarbamoyl ((C₁-C₄)alkylNHCO—), (C₁-C₄)alkylsulphonyl ((C₁-C₄)alkylSO₂—), C₁-C₄ alkoxy, (C₁-C₄)alkylsulphonamido ((C₁-C₄)alkylSO₂NH—), (C₁-C₄)alkylsulphamoyl ((C₁-C₄)alkylNHSO₂—) and onium Z radicals;
   NR′₃R′₄;
   a carboxyl radical;
   a C₁-C₄ alkoxycarbonyl radical;
   a (C₁-C₄)alkylamido radical ((C₁-C₄)alkylCONH—);
   a (C₁-C₄)alkylsulphonyl radical (alkylSO₂—);
   an alkylsulphonamido radical ((C₁-C₄)alkylSO₂NH—);
   a hydroxyl radical;
   a C₁-C₄ alkoxy radical;
   a C₂-C₄ hydroxyalkoxy radical;
   a (C₁-C₄)alkylcarbamoyl radical ((C₁-C₄)alkylNHCO—);
   (C₁-C₄)alkylsulphamoyl ((C₁-C₄)alkyl-NH—SO₂—);
   a C₁-C₄ thioether radical;
   a sulphonic radical (SO₃H), which may be in salt form;
   an onium radical Z, R′₃ and R′₄, which may be identical or different, represent a hydrogen atom; a C₁-C₄ alkyl radical optionally substituted with one or more radicals chosen from hydroxyl, C₁-C₄ alkoxy, amino, mono- or dialkylamino, (C₁-C₄)alkylCO—, (C₁-C₄)alkylNHCO— and (C₁-C₄)alkylSO₂— radicals,
   n is an integer between 1 and 8,
   m is an integer between 0 and 3, preferably from 0 to 2,
   Y represents an oxygen atom, a radical CR′, a radical NR′₅ or a radical NR′₆R′₇ with
      R′₅ which represents a hydrogen atom; a linear or branched C₁-C₁₀ hydrocarbon-based chain, which may be saturated or unsaturated, one or more of the carbon atoms of the carbon-based chain of which may be replaced with an oxygen, nitrogen or sulphur atom or with an SO₂ group; R′₅ not comprising a peroxide bond or diazo or nitroso radicals, and R′₅ not being directly linked to the nitrogen atom via an oxygen, sulphur or nitrogen atom,
      R′₆ and R′₇ which represent, independently, a linear or branched C₁-C₁₀ hydrocarbon-based chain, which may be saturated or unsaturated, one or more carbon atoms of the carbon-based chain of which may be replaced with an oxygen, nitrogen or sulphur atom or with an SO₂ group; R′₆ and R′₇ not comprising a peroxide bond or diazo or nitroso radicals, and R′₆ and R′₇ not being directly linked to the nitrogen atom via an oxygen, sulphur or nitrogen atom,
W₁ represents an aromatic heterocyclic radical chosen from the following radicals 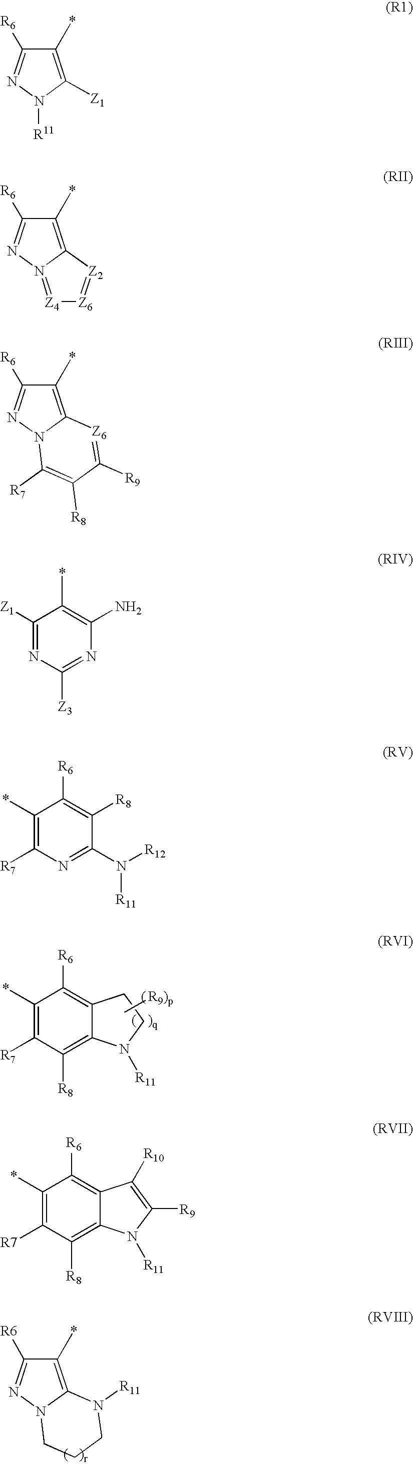
Z₁ and Z₃ represent, independently of each other, a hydroxyl or NR₁₁R₁₂ radical,
Z₂, Z₄ and Z₆ represent, independently of each other, a nitrogen atom or a radical CR₁₂ or NR₁₁, with the proviso that at least one of them represents a radical CR₁₂ and that there cannot be more than three contiguous nitrogen atoms,
Z₈ represents a nitrogen atom or a radical CR₁₅,
R₆, R₇, R₈, R₉, R₁₀, R₁₁, R₁₂ and R₁₅ represent, independently of each other:
   a hydrogen atom,
   a linear or branched C₁-C₁₀ hydrocarbon-based chain, which can form one or more 4- to 8-membered carbon-based rings, and which may be saturated or unsaturated, one or more carbon atoms of the carbon-based chain of which may be replaced with an oxygen, nitrogen or sulphur atom or with an SO₂ group; the radicals R₆ to R₁₂ and R₁₅ not comprising a peroxide bond or diazo or nitroso radicals, and the radical R₁₁ not being directly linked to the nitrogen atom via an oxygen, sulphur or nitrogen atom,
   p may take the values 4 to 8,
   q may take the values 1 to 3, and
   r may take the values 0 to 2,
   * indicates the point of attachment of W₁ in formula (I).

According to one particular embodiment, the dye composition according to the present invention comprises, in a suitable medium, a compound of formula (I) below or an addition salt thereof: 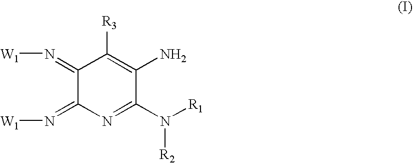
in which
R₃ represents:
   a hydrogen atom,
   a linear or branched C₁-C₁₀ hydrocarbon-based chain, which can form one or more 4- to 8-membered carbon-based rings, and which may be saturated or unsaturated, one or more carbon atoms of the carbon-based chain of which may be replaced with an oxygen, nitrogen or sulphur atom or with an SO₂ group, and the carbon atoms of which may be, independently of each other, substituted with one or more halogen atoms or hydroxyl, amino, carboxyl, sulphonic or thiol radicals; R₃ not comprising a peroxide bond or diazo or nitroso radicals,
   NR′₁R′₂, R′₁ and R′₂ being as defined for R₁ and R₂
R₁ and R₂ represent, independently of each other:
   a hydrogen atom
   a linear or branched C₁-C₁₀ hydrocarbon-based chain, which can form one or more 4- to 8-membered carbon-based rings, and which may be saturated or unsaturated, one or more carbon atoms of the carbon-based chain of which may be replaced with an oxygen, nitrogen or sulphur atom or with an SO₂ group, and the carbon atoms of which may be, independently of each other, substituted with one or more halogen atoms or hydroxyl, amino, carboxyl, sulphonic or thiol radicals; R₁ and R₂ not comprising a peroxide bond or diazo or nitroso radicals, and R₁ and R₂ not being linked directly to the nitrogen atom via an oxygen, sulphur or nitrogen atom or SO₂,
   an onium radical Z, or
R₁ and R₂ form, together with the nitrogen atom to which they are attached, a ring of formula (II): 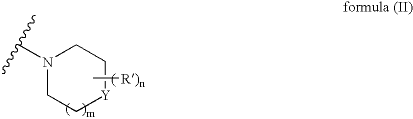
   in which:
R′ represents:
   a hydrogen atom;
   a halogen atom such as fluorine, chlorine or bromine;
   a C₁-C₄ alkyl radical optionally substituted with one or more radicals chosen from hydroxyl, carboxyl, C₁-C₄ alkoxycarbonyl, (C₁-C₄)alkylamido ((C₁-C₄)alkylCONH—), (C₁-C₄)alkylcarbamoyl ((C₁-C₄)alkylNHCO—), (C₁-C₄)alkylsulphonyl ((C₁-C₄)alkylSO₂—), C₁-C₄ alkoxy, (C₁-C₄)alkylsulphonamido((C₁-C₄)alkylSO₂NH—), (C₁-C₄)alkylsulphamoyl ((C₁-C₄)alkylNHSO₂—), and onium Z radicals,
   NR′₃R′₄;
   a carboxyl radical;
   a C₁-C₄ alkoxycarbonyl radical;
   a (C₁-C₄)alkylamido radical ((C₁-C₄)alkylCONH—);
   a (C₁-C₄)alkylsulphonyl radical (alkylSO₂—);
   an alkylsulphonamido radical ((C₁-C₄)alkylSO₂NH—);
   a hydroxyl radical;
   a C₁-C₄ alkoxy radical;
   a C₂-C₄ hydroxyalkoxy radical;
   a (C₁-C₄)alkylcarbamoyl radical ((C₁-C₄)alkylNHCO—);
   (C₁-C₄)alkylsulphamoyl ((C₁-C₄)alkyl-NH—SO₂—);
   a C₁-C₄ thioether radical;
   a sulphonic radical (SO₃H) which may be in salt form;
   an onium radical Z;
R′₃ and R′₄, which may be identical or different, represent a hydrogen atom; a C₁-C₄ alkyl radical optionally substituted with one or more radicals chosen from hydroxyl, C₁-C₄ alkoxy, amino, monoalkylamino, dialkylamino, (C₁-C₄)alkylCO—, (C₁-C₄)alkylNHCO— and (C₁-C₄)alkylSO₂— radicals,
   n is an integer between 1 and 8,
   m is an integer between 0 and 3 and preferably between 0 and 2,
   Y represents an oxygen atom, a radical CR′, a radical NR′ ₅ or a radical NR′₆R′₇, with
      R′₅ which represents a hydrogen atom; a linear or branched C₁-C₁₀ hydrocarbon-based chain, which may be saturated or unsaturated, one or more carbon atoms of the carbon-based chain of which may be replaced with an oxygen, nitrogen or sulphur atom or with an SO₂ group, and the carbon atoms of which may be, independently of each other, substituted with one or more halogen atoms or hydroxyl, amino, carboxyl, sulphonic or thiol radicals;
      R′₅ not comprising a peroxide bond or diazo or nitroso radicals, and R′₅ not being linked directly to the nitrogen atom via an oxygen, sulphur or nitrogen atom,
      R′₆ and R′₇ which independently represent a linear or branched C₁-C₁₀ hydrocarbon-based chain, which may be saturated or unsaturated, one or more carbon atoms of the carbon-based chain of which may be replaced with an oxygen, nitrogen or sulphur atom or with an SO₂ group, and the carbon atoms of which may be, independently of each other, substituted with one or more halogen atoms or hydroxyl, amino, carboxyl, sulphonic or thiol radicals;
      R′₆ and R′₇ not comprising a peroxide bond or diazo or nitroso radicals, and R′₆ and R′₇ not being linked directly to the nitrogen atom via an oxygen, sulphur or nitrogen atom,
W₁ represents an aromatic heterocyclic radical chosen from the following radicals 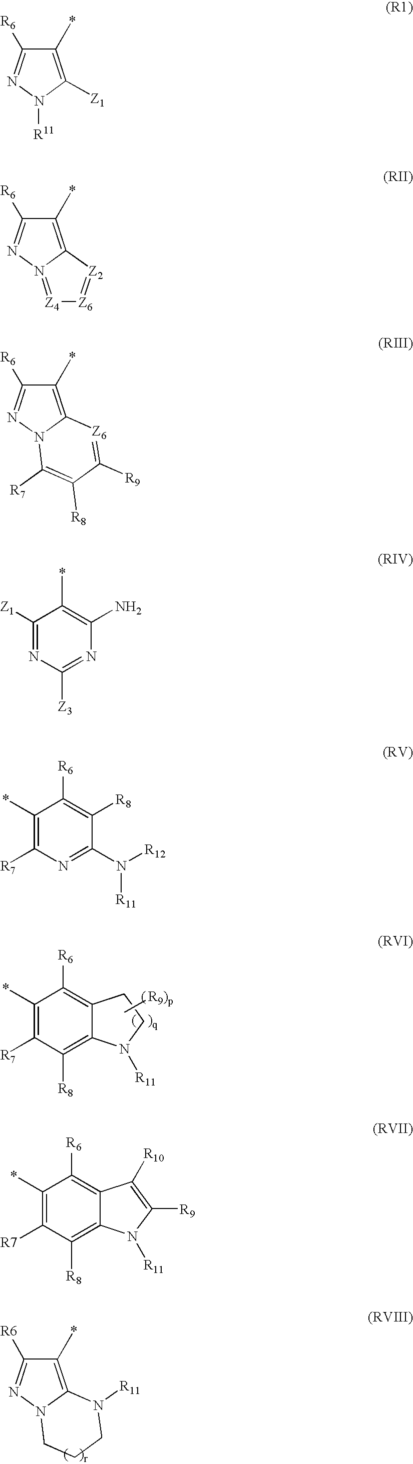
Z₁ and Z₃ represent, independently of each other, a hydroxyl radical or a radical NR₁₁R₁₂;
Z₂, Z₄ and Z₆ represent, independently of each other, a nitrogen atom or a radical CR₁₂ or NR₁₁, with the proviso that at least one of them represents a radical CR₁₂ and that there cannot be more than three contiguous nitrogen atoms,
Z₈ represents a nitrogen atom or a radical CR₁₅;
R₆, R₇, R₈, R₉, R₁₀, R₁₁, R₁₂ and R₁₅ represent, independently of each other:
   a hydrogen atom,
   a linear or branched C₁-C₁₀ hydrocarbon-based chain, which can form one or more 4- to 8-membered carbon-based rings, and which may be saturated or unsaturated, one or more carbon atoms of the carbon-based chain of which may be replaced with an oxygen, nitrogen or sulphur atom or with an SO₂ group, and the carbon atoms of which may be, independently of each other, substituted with one or more halogen atoms or hydroxyl, amino, carboxyl, sulphonic or thiol radicals; the radicals R₆ to R₁₂ and R₁₅ not comprising a peroxide bond or diazo or nitroso radicals and the radical R₁₁ not being linked directly to the nitrogen atom via an oxygen, sulphur or nitrogen atom,
   p can take the values 4 to 8,
   q can take the values 1 to 3, and
   r can take the values 0 to 2,
   * indicates the point of attachment of W₁ in formula (I).

According to another particular embodiment, the dye composition according to the present invention comprises, in a suitable medium, a compound of formula (I) below or an addition salt thereof: 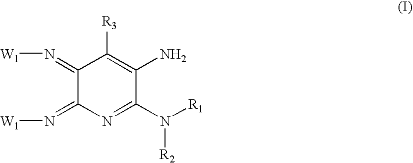
in which
R₃ represents:
   a hydrogen atom,
   a linear or branched C₁-C₁₀ hydrocarbon-based chain, which can form one or more 4- to 8-membered carbon-based rings, and which may be saturated or unsaturated, one or more carbon atoms of the carbon-based chain of which may be replaced with an oxygen, nitrogen or sulphur atom or with an SO₂ group, and the carbon atoms of which may be, independently of each other, substituted with one or more halogen atoms or hydroxyl, amino, C₁-C₂ (di)alkylamino, C₁-C₂ alkoxy, carboxyl, sulphonic or thiol radicals; R₃ not comprising a peroxide bond or diazo or nitroso radicals,
   NR′₁R′₂, R′₁ and R′₂ being as defined for R₁ and R₂
R₁ and R₂ represent, independently of each other:
   a hydrogen atom
   a linear or branched C₁-C₁₀ hydrocarbon-based chain, which can form one or more 4- to 8-membered carbon-based rings, and which may be saturated or unsaturated, one or more carbon atoms of the carbon-based chain of which may be replaced with an oxygen, nitrogen or sulphur atom or with an SO₂ group, and the carbon atoms of which may be, independently of each other, substituted with one or more halogen atoms or hydroxyl, amino, C₁-C₂ (di)alkylamino, C₁-C₂ alkoxy, carboxyl, sulphonic or thiol radicals; R₁ and R₂ not comprising a peroxide bond or diazo or nitroso radicals, and R₁ and R₂ not being linked directly to the nitrogen atom via an oxygen, sulphur or nitrogen atom or SO₂,
   an onium radical Z, or
R₁ and R₂ form, together with the nitrogen atom to which they are attached, a ring of formula (II): 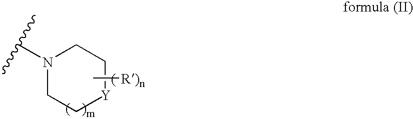
   in which:
R′ represents:
   a hydrogen atom;
   a halogen atom such as fluorine, chlorine or bromine;
   a C₁-C₄ alkyl radical optionally substituted with one or more radicals chosen from hydroxyl, carboxyl, C₁-C₄ alkoxycarbonyl, (C₁-C₄)alkylamido ((C₁-C₄)alkylCONH—), (C₁-C₄)alkylcarbamoyl ((C₁-C₄)alkylNHCO—), (C₁-C₄)alkylsulphonyl ((C₁-C₄)alkylSO₂—), C₁-C₄ alkoxy, (C₁-C₄)alkylsulphonamido((C₁-C₄)alkylSO₂NH—), (C₁-C₄)alkylsulphamoyl ((C₁-C₄)alkylNHSO₂—), and onium Z radicals,
   NR′₃R′₄;
   a carboxyl radical;
   a C₁-C₄ alkoxycarbonyl radical;
   a (C₁-C₄)alkylamido radical ((C₁-C₄)alkylCONH—);
   a (C₁-C₄)alkylsulphonyl radical (alkylSO₂—);
   an alkylsulphonamido radical ((C₁-C₄)alkylSO₂NH—);
   a hydroxyl radical;
   a C₁-C₄ alkoxy radical;
   a C₂-C₄ hydroxyalkoxy radical;
   a (C₁-C₄)alkylcarbamoyl radical ((C₁-C₄)alkylNHCO—);
   (C₁-C₄)alkylsulphamoyl ((C₁-C₄)alkyl-NH—SO₂—);
   a C₁-C₄ thioether radical;
   a sulphonic radical (SO₃H) which may be in salt form;
   an onium radical Z;
R′₃ and R′₄, which may be identical or different, represent a hydrogen atom; a C₁-C₄ alkyl radical optionally substituted with one or more radicals chosen from hydroxyl, C₁-C₄ alkoxy, amino, monoalkylamino, dialkylamino, (C₁-C₄)alkylCO—, (C₁-C₄)alkylNHCO— and (C₁-C₄)alkylSO₂— radicals,
   n is an integer between 1 and 8,
   m is an integer between 0 and 3 and preferably between 0 and 2,
   Y represents an oxygen atom, a radical CR′, a radical NR′₅ or a radical NR′₆R′₇, with
      R′₅ which represents a hydrogen atom; a linear or branched C₁-C₁₀ hydrocarbon-based chain, which may be saturated or unsaturated, one or more carbon atoms of the carbon-based chain of which may be replaced with an oxygen, nitrogen or sulphur atom or with an SO₂ group, and the carbon atoms of which may be, independently of each other, substituted with one or more halogen atoms or hydroxyl, amino, carboxyl, sulphonic or thiol radicals;
      R′₅ not comprising a peroxide bond or diazo or nitroso radicals, and R′₅ not being linked directly to the nitrogen atom via an oxygen, sulphur or nitrogen atom,
      R′₆ and R′₇ which independently represent a linear or branched C₁-C₁₀ hydrocarbon-based chain, which may be saturated or unsaturated, one or more carbon atoms of the carbon-based chain of which may be replaced with an oxygen, nitrogen or sulphur atom or with an SO₂ group, and the carbon atoms of which may be, independently of each other, substituted with one or more halogen atoms or hydroxyl, amino, carboxyl, sulphonic or thiol radicals;
      R′₆ and R′₇ not comprising a peroxide bond or diazo or nitroso radicals, and R′₆ and R′₇ not being linked directly to the nitrogen atom via an oxygen, sulphur or nitrogen atom,
W₁ represents an aromatic heterocyclic radical chosen from the following radicals 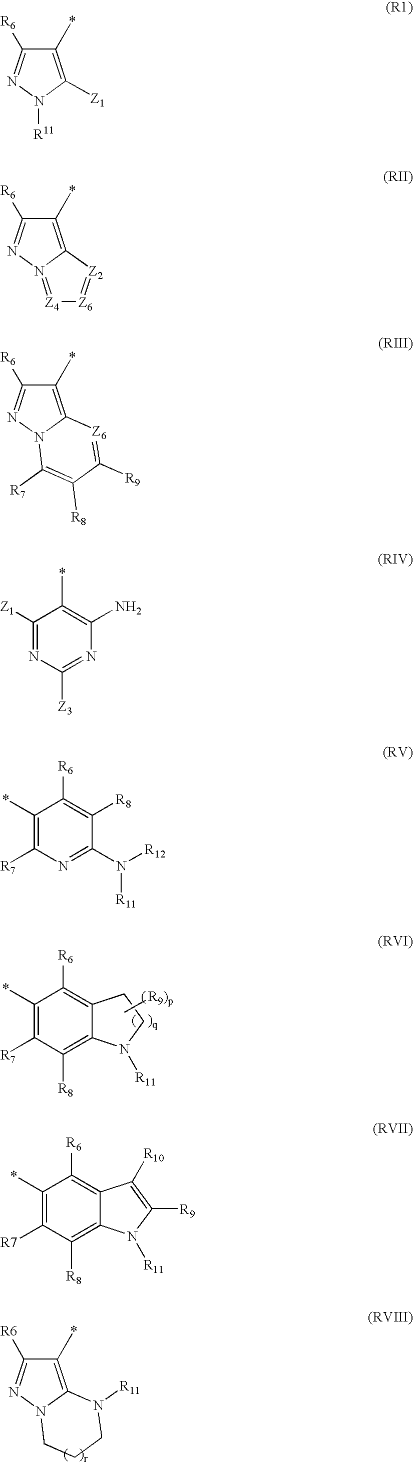
Z₁ and Z₃ represent, independently of each other, a hydroxyl radical or a radical NR₁₁R₁₂;
Z₂, Z₄ and Z₆ represent, independently of each other, a nitrogen atom or a radical CR₁₂ or NR₁₁, with the proviso that at least-one of them represents a radical CR₁₂ and that there cannot be more than three contiguous nitrogen atoms,
Z₈ represents a nitrogen atom or a radical CR₁₅;
R₆, R₇, R₈, R₉, R₁₀, R₁₁, R₁₂ and R₁₅ represent, independently of each other:
   a hydrogen atom,
   a linear or branched C₁-C₁₀ hydrocarbon-based chain, which can form one or more 4- to 8-membered carbon-based rings, and which may be saturated or unsaturated, one or more carbon atoms of the carbon-based chain of which may be replaced with an oxygen, nitrogen or sulphur atom or with an SO₂ group, and the carbon atoms of which may be, independently of each other, substituted with one or more halogen atoms or hydroxyl, amino, carboxyl, sulphonic or thiol radicals; the radicals R₆ to R₁₂ and R₁₅ not comprising a peroxide bond or diazo or nitroso radicals and the radical R₁₁ not being linked directly to the nitrogen atom via an oxygen, sulphur or nitrogen atom,
   p can take the values 4 to 8,
   q can take the values 1 to 3, and
   r can take the values 0 to 2,
   * indicates the point of attachment of W₁ in formula (I).

When, in the definition of Z₂, Z₄ and Z₆ which represent, independently of each other, a nitrogen atom or a radical CR₁₂ or NR₁₁, it is specified that there cannot be more than 3 contiguous nitrogen atoms, this means that it is not possible, for example, to obtain the following structure

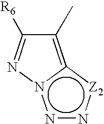

A subject of the present invention is also a dye composition containing, in a suitable medium, at least one direct dye of the present invention. The composition of the present invention is particularly useful for dyeing keratin fibres, in particular human keratin fibres.

In the context of the present invention, the compounds of formula (I) are not only those described by formula (I), but any other tautomeric form, for instance the following tautomeric form:

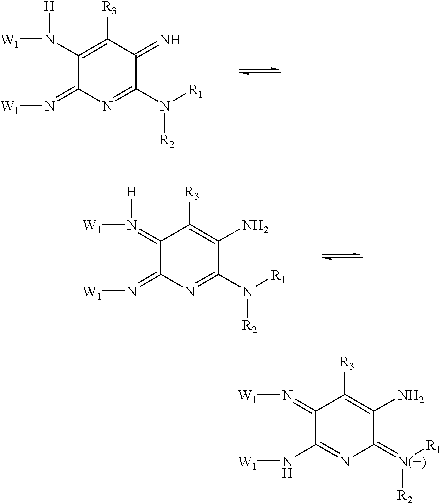

In the examples that are given in the description hereinbelow, only one of these tautomeric forms will be indicated.

In formula (I), R₃ is preferably chosen from a hydrogen atom and a C₁-C₄ alkyl radical optionally substituted with one or more radicals chosen from hydroxyl, C₁-C₂ alkoxy, amino, C₁-C₂ (mono)- or (di)alkylamino, 2-hydroxyethyl and 2-aminoethyl radicals. By way of example, R₃ represents a hydrogen atom or a methyl, ethyl or 2-hydroxyethyl radical and even more preferably: a hydrogen atom or a methyl radical.

The radicals R₁ and R₂ are preferably chosen, separately, from a hydrogen atom and a C₁-C6 alkyl radical optionally substituted with a hydroxyl, alkoxy, amino or C₁-C₄ (mono)- or (di)alkylamino. By way of example, R₁ and R₂ are chosen from a hydrogen atom, a methyl, ethyl, hydroxyethyl, propyl, etc. radical.

When R₁ and R₂ form, with the nitrogen atom to which they are attached, a 5- or 8-membered heterocycle, this heterocycle is preferably chosen from pyrrolidine, piperidine, homopiperidine, piperazine, homopiperazine and optionally substituted diazepane heterocycles. By way of example, the heterocycle is chosen from pyrrolidine, 3-hydroxypyrrolidine, 3-aminopyrrolidine, 3-N,N-dimethylaminopyrrolidine, 3-acetamidopyrrolidine, 3-(methylsulphonylamino)pyrrolidine, proline, 3-hydroxyproline, piperidine, hydroxypiperidine, homopiperidine, diazepane, N-methylhomopiperazine and N-β-hydroxyethylhomopiperazine, and the addition salts thereof. Preferably, R₁ and R₂ form, with the nitrogen atoms to which they are attached, an optionally substituted pyrrolidine ring.

According to one particular embodiment, the compound of formula (I) is a cationic compound substituted with at least one onium radical Z, ie a cationic radical of the quaternary ammonium type.

The onium radical Z may be represented by formula (III) below 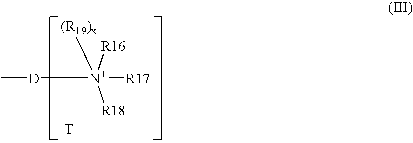
in which
D is a covalent bond or a linear or branched C₁-C₁₄ alkylene chain which may contain one or more hetero atoms chosen from oxygen, sulphur and nitrogen, SO₂ or one or more ketone functions, the chain possibly being substituted with one or more hydroxyl, C₁-C₆ alkoxy, amino or C₁-C₄ (mono)- or (di)alkylamino radicals,
R₁₆, R₁₇ and R₁₈, taken separately, represent a C₁-C₁₅ alkyl radical; a C₁-C₆ monohydroxyalkyl radical; a C₂-C₆ polyhydroxyalkyl radical; a (C₁-C₆)alkoxy(C₁-C₆)alkyl radical; an aryl radical; a benzyl radical; a C₁-C₆ amidoalkyl radical; a tri(C₁-C₆)alkylsilane(C₁-C₆)alkyl radical; a C₁-C₆ aminoalkyl radical; a C₁-C₆ aminoalkyl radical in which the amine is mono- or disubstituted with a C₁-C₄ alkyl, (C₁-C₆)alkylcarbonyl, amido or (C₁-C₆)alkylsulphonyl radical; a carbamyl(C₁-C₆)alkyl radical; a (C₁-C₆)alkylcarboxy (C₁-C₆)alkyl radical; a (C₁-C₆)alkylcarbonyl (C₁-C₆)alkyl radical; an N—(C₁-C₆)alkylcarbamyl (C₁-C₆)alkyl radical;
R₁₆, R₁₇ and R₁₈ together, in pairs, form, with the nitrogen atom to which they are attached, a 4-, 5-, 6- or 7-membered carbon-based saturated ring which may contain one or more hetero atoms, the cationic ring possibly being substituted with a halogen atom, a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri (C₁-C₆)alkylsilane (C₁-C₆)alkyl radical, an amido radical, a carboxyl radical, a C₁-C₆ alkylcarbonyl radical, a thio radical, a C₁-C₆ thioalkyl radical, a (C₁-C₆)alkylthio radical, an amino radical or an amino radical mono- or disubstituted with a (C₁-C₆)alkyl, (C₁-C₆)alkylcarbonyl, amido or (C₁-C₆)alkylsulphonyl radical;
R₁₉ represents a C₁-C₆ alkyl radical; a C₁-C₆ monohydroxyalkyl radical; a C₂-C₆ polyhydroxyalkyl radical; an aryl radical; a benzyl radical; a C₁-C₆ aminoalkyl radical; a C₁-C₆ aminoalkyl radical in which the amine is mono- or disubstituted with a (C₁-C₆)alkyl, (C₁-C₆)alkylcarbonyl, amido or (C₁-C₆)alkylsulphonyl radical; a carboxy(C₁-C₆)alkyl radical; a carbamyl(C₁-C₆)alkyl radical; a C₁-C₆ trifluoroalkyl radical; a tri(C₁-C₆)alkylsilane(C₁-C₆)alkyl radical; a C₁-C₆ sulphonamidoalkyl radical; a (C₁-C₆)alkylcarboxy(C₁-C₆)alkyl radical; a (C₁-C₆)alkylsulphinyl(C₁-C₆)alkyl radical; a (C₁-C₆)alkylsulphonyl (C₁-C₆)alkyl radical; a (C₁-C₆)alkylcarbonyl (C₁-C₆)alkyl radical; an N—(C₁-C₆)alkylcarbamyl (C₁-C₆)alkyl radical; an N—(C₁-C₆)alkylsulphonamido (C₁-C₆)alkyl radical;
x is 0 or 1,
   when x=0, then linker arm D is attached to the nitrogen atom bearing the radicals R₁₆ to R₁₈,
   when x=1, then two of the radicals R₁₆ to R₁₈ form, together with the nitrogen atom to which they are attached, a 5-, 6- or 7-membered saturated ring and the linker arm D is linked to a carbon atom of the saturated ring;
T is a counterion.

According to a first variant of formula (III), when x is equal to 0 and R₁₆, R₁₇ and R₁₈, separately, are preferably chosen from a C₁-C₆ alkyl radical, a C₁-C₄ monohydroxyalkyl radical, a C₂-C₄ polyhydroxyalkyl radical, a (C₁-C₆)alkoxy(C₁-C₄)alkyl radical, a C₁-C₆ amidoalkyl radical or a tri (C₁-C₆)alkylsilane (C₁-C₆)alkyl radical.

According to a second variant of formula (III), when x is equal to 0 and R₁₆ and R₁₇ together form an azetidine, pyrrolidine, piperidine, homopiperidine, piperazine, homopiperazine or morpholine ring, then R₁₈ is chosen from a C₁-C₆ alkyl radical; a C₁-C₆ monohydroxyalkyl radical; a C₂-C₆ polyhydroxyalkyl radical; a C₁-C₆ aminoalkyl radical; an aminoalkyl radical in which the amine is mono- or disubstituted with a (C₁-C₄)alkyl, (C₁-C₆)alkylcarbonyl, amido or (C₁-C₆)alkylsulphonyl radical; a C₁-C₆ carbamylalkyl radical; a tri(C₁-C₆)alkylsilane(C₁-C₆)alkyl radical; a (C₁-C₆)alkylcarboxy(C₁-C₆)alkyl radical; a (C₁-C₆)alkylcarbonyl(C₁-C₆)alkyl radical; an N—(C₁-C₆)alkylcarbamyl (C₁-C₆)alkyl radical.

In formula (III), when x is equal to 1, R₁₉ is preferably chosen from a C₁-C₆ alkyl radical; a C₁-C₆ monohydroxyalkyl radical; a C₂-C₆ polyhydroxyalkyl radical; a C₁-C₆ aminoalkyl radical; a C₁-C₆ aminoalkyl radical in which the amine is mono- or disubstituted with a (C₁-C₆)alkyl, (C₁-C₆)alkylcarbonyl, amido or (C₁-C₆)alkylsulphonyl radical; a C₁-C₆ carbamylalkyl radical; a tri(C₁-C₆)alkylsilane(C₁-C₆)alkyl radical; a (C₁-C₆)alkylcarboxy(C₁-C₆)alkyl radical; a (C₁-C₆)alkylcarbonyl(C₁-C₆)alkyl radical; an N—(C₁-C₆)alkylcarbamyl(C₁-C₆)alkyl radical; R₁₆ and R₁₇ together form an azetidine, pyrrolidine, piperidine, homopiperidine, piperazine, homopiperazine or morpholine ring, and R₁₈ is then chosen from a C₁-C₆ alkyl radical; a C₁-C₆ monohydroxyalkyl radical; a C₂-C₆ polyhydroxyalkyl radical; a C₁-C₆ aminoalkyl radical; a C₁-C₆ aminoalkyl radical in which the amine is mono- or disubstituted with a (C₁-C₄)alkyl, (C₁-C₆)alkylcarbonyl, amido or (C₁-C₆)alkylsulphonyl radical; a C₁-C₆ carbamylalkyl radical; a tri (C₁-C₆)alkylsilane (C₁-C₆)alkyl radical; a (C₁-C₆)alkylcarboxy(C₁-C₆)alkyl radical; a (C₁-C₆)alkylcarbonyl(C₁-C₆)alkyl radical; an N—(C₁-C₆)alkylcarbamyl (C₁-C₆)alkyl radical.

According to one preferred embodiment, x is equal to 0, and R₁₆, R₁₇ and R₁₈ are alkyl radicals.

In formula (III), D is preferably a covalent bond or a C₁-C₆ alkylene chain which may be substituted.

The onium radical Z may also be represented by formula (IV) 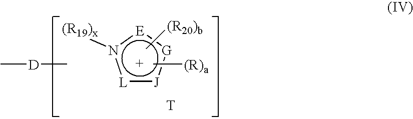
in which
D is as defined above,
the ring members E, G, J and L, which may be identical or different, represent a carbon, oxygen, sulphur or nitrogen atom to form a pyrazole, imidazole, triazole, oxazole, isoxazole, thiazole or isothiazole ring,
a is an integer between 0 and 3 inclusive;
b is an integer between 0 and 1 inclusive;
a+b is an integer between 2 and 4,
R, which may be identical or different, represent a hydrogen or halogen atom, a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri (C₁-C₆)alkylsilane (C₁-C₆)alkyl radical, an amido radical, a carboxyl radical, a C₁-C₆ alkylcarbonyl radical, a thio radical, a C₁-C₆ thioalkyl radical, a (C₁-C₆)alkylthio radical, an amino radical, an amino radical mono- or disubstituted with a (C₁-C₆)alkyl, (C₁-C₆)alkylcarbonyl, amido or (C₁-C₆)alkylsulphonyl radical; a C₁-C₆ monohydroxyalkyl radical or a C₂-C₆ polyhydroxyalkyl radical; a benzyl radical; a phenyl radical optionally substituted with one or more radicals chosen from methyl, hydroxyl, amino and methoxy radicals; it being understood that the radicals R are borne by a carbon atom,
R₂₀ represents a C₁-C₆ alkyl radical, a C₁-C₆ monohydroxyalkyl radical, a C₂-C₆ polyhydroxyalkyl radical, a tri (C₁-C₆)alkylsilane (C₁-C₆)alkyl radical, a (C₁-C₆)alkoxy (C₁-C₆)alkyl radical, a C₁-C₆ carbamylalkyl radical, a (C₁-C₆)alkylcarboxy-(C₁-C₆)alkyl radical or a benzyl radical; it being understood that the radical R₂₀ is borne by a nitrogen atom,
R₁₉ is as defined above,
x is equal to 0 or 1,
   when x=0, the linker arm D is attached to the nitrogen atom,
   when x=1, the linker arm D is attached to one of the ring members E, G, J or L when E, G, J or L represents a carbon atom,
T is a counterion.

Preferably, the ring members E, G, J and L form an imidazole, pyrazole, oxazole, thiazole or triazole ring.

According to one particular embodiment of formula (III), x is equal to 0, and D is a covalent bond or a C₁-C₄ alkylene chain which may be substituted.

The onium radical Z may be represented by formula (V) 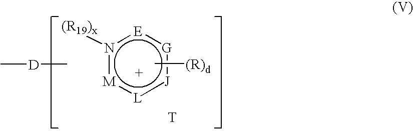
in which
D, R and R₁₉ are as defined above,
the ring members E, G, J, L and M, which may be identical or different, represent a carbon or nitrogen atom and form a ring chosen from pyridine, pyrimidine, pyrazine, triazine and pyridazine rings,
d is an integer between 3 and 5 inclusive,
x is equal to 0 or 1,
   when x=0, the linker arm D is attached to the nitrogen atom,
   when x=1, the linker arm D is attached to one of the ring members E, G, J, L or M, when E, G, J, L or M represents a carbon atom,
T represents a counterion.

Preferably, the ring members E, G, J, L and M form, with the nitrogen of the ring, a ring chosen from pyridine, pyrimidine, pyridazine and pyrazine rings.

According to one variant of formula (V), x is equal to 0 and R is chosen from a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri(C₁-C₆)alkylsilane(C₁-C₆)alkyl radical, an amido radical, a C₁-C₆ alkylcarbonyl radical, an amino radical, an amino radical mono- or disubstituted with a (C₁-C₆)alkyl, (C₁-C₆)alkylcarbonyl, amido or (C₁-C₆)alkylsulphonyl radical; a C₁-C₆ monohydroxyalkyl radical or a C₂-C₆ polyhydroxyalkyl radical; it being understood that the radicals R are borne by a carbon atom.

According to another variant of formula (V), x is equal to 1, R₁₉ is chosen from a C₁-C₆ alkyl radical; a C₁-C₆ monohydroxyalkyl radical; a C₂-C₆ polyhydroxyalkyl radical; a C₁-C₆ aminoalkyl radical, a C₁-C₆ aminoalkyl radical in which the amine is mono- or disubstituted with a (C₁-C₆)alkyl, (C₁-C₆)alkylcarbonyl, amido or (C₁-C₆)alkylsulphonyl radical; a C₁-C₆ carbamylalkyl radical; a tri(C₁-C₆)alkylsilane(C₁-C₆)-alkyl radical; a (C₁-C₆)alkylcarbonyl(C₁-C₆)alkyl radical; an N—(C₁-C₆)alkylcarbamyl (C₁-C₆)alkyl radical; R is chosen from a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri (C₁-C₆)alkylsilane (C₁-C₆)alkyl radical, an amido radical, a C₁-C₆ alkylcarbonyl radical, an amino radical or an amino radical mono- or disubstituted with a (C₁-C₆)alkyl, (C₁-C₆)alkylcarbonyl, amido or (C₁-C₆)alkylsulphonyl radical.

In one preferred embodiment, R₁₉ is a C₁-C₄ alkyl radical which may be substituted, and R is a hydrogen radical or a C₁-C₄ alkyl radical which may be substituted.

When the compound of formula (I) is a cationic compound substituted with a radical Z, preferably, at least one of the radicals R₁ and R₂ represents an onium radical Z. According to one particular embodiment, R₁ and R₂ form a ring of formula (II) in which R′ is an onium radical Z, preferably with Y equal to NR′₆R′₇.

In formula (I), W₁ in particular represents 5-aminopyrazole, 5-hydroxypyrazole, pyrazolo[1,5-b]pyridine, pyrazolo[1,5-a]pyrimidine, pyrazolo[3,2-c]triazole, pyrazolo[1,5-b]triazole, aminopyrimidine, triaminopyrimidine, hydroxyaminopyrimidine, 2-aminopyridine, indoline and indole radicals.

According to one particular embodiment, W₁ is chosen from the 5-aminopyrazole and 5-hydroxypyrazole radicals of formula (R1).

In this case, W₁ is preferably chosen from 5-aminopyrazole and 5-hydroxypyrazole radicals in which R₆ and R₁₁, which may be identical or different, are chosen from a hydrogen atom; a linear or branched C₁-C₁₀ hydrocarbon-based chain, which can form one or more 4- to 8-membered carbon-based rings, and which may be saturated or unsaturated, at one or more of the carbon atoms of the carbon-based chain of which may be replaced with an oxygen, nitrogen or sulphur atom or with an SO₂ group,,and the carbon atoms of which may be, independently of each other, substituted with one or more halogen atoms or hydroxyl, amino, carboxyl, sulphonic or thiol radicals; the radicals R₆ to R₁₂ not comprising a peroxide bond or diazo or nitroso radicals and the radical R₁₁ not being linked directly to the nitrogen atom via an oxygen, sulphur or nitrogen atom.

According to one particularly preferred embodiment of (R₁), R₆ and R₁₁ are chosen, independently, from a hydrogen atom and a linear or branched C₁-C₄ hydrocarbon-based chain, which can form one or more 5- or 6-membered carbon-based rings, and which may be saturated or unsaturated, the carbon atoms of which may be, independently of each other, substituted with one or more halogen atoms or hydroxyl or amino radicals.

According to another particular embodiment, W₁ represents 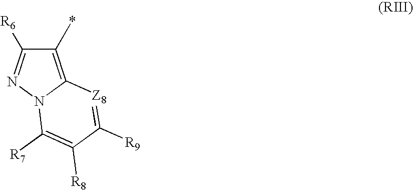
R₆, R₇, R₈, R₉ and Z₈ being as defined above.

In this particular case, W₁ may be a pyrazolo[1,5-b]pyridine radical in which R_{6,} R₇, R₈, R₉ and R₁₅, which may be identical or different, are chosen from
a hydrogen atom,
a linear or branched C₁-C₁₀ hydrocarbon-based chain, which may form one or more 4- to 8-membered carbon-based rings, and which may be saturated or unsaturated, one or more carbon atoms of the carbon-based chain of which may be replaced with an oxygen, nitrogen or sulphur atom or with an SO₂ group, and the carbon atoms of which may be, independently of each other, substituted with one or more halogen atoms or hydroxyl, amino, carboxyl, sulphonic or thiol radicals; the radicals not comprising a peroxide bond or diazo or nitroso radicals,
hydroxyl or amino radicals, the amine possibly being substituted with a linear or branched C₁-C₄ hydrocarbon-based chain, which can form one or more 5- or 6-membered carbon-based rings, and which may be saturated or unsaturated, the carbon atoms may be, independently of each other, substituted with one or more halogen atoms or hydroxyl or amino radicals.

In this particular case, W, may also be a pyrazolo[1,5-b]pyridine radical in which R₆, R₇, R₈, R₉ and R₁₅, which may be identical or different, are chosen from:
a hydrogen atom,
a linear or branched C₁-C₁₀ hydrocarbon-based chain, which can form one or more 4- to 8-membered carbon-based rings, and which may be saturated or unsaturated, one or more carbon atoms of the carbon-based chain of which may be replaced with an oxygen, nitrogen or sulphur atom or with an SO₂ group, and the carbon atoms of which may be, independently of each other, substituted with one or more halogen atoms or hydroxyl, amino, carboxyl, sulphonic or thiol radicals; the radicals not comprising a peroxide bond or diazo or nitroso radicals,
hydroxyl or amino radicals, the amine possibly being substituted with a linear or branched C₁-C₄ hydrocarbon-based chain, which can form one or more 6-membered carbon-based rings, and which may be saturated or unsaturated, the carbon atoms may be, independently of each other, substituted with one or more halogen atoms or hydroxyl or amino radicals.

In this particular case, W₁ may also be a pyrazolo[1,5-b]pyridine radical in which R₆, R₇, R₈, R₉ and R₁₅, which may be identical or different, are chosen from:
a hydrogen atom,
a linear or branched C₁-C₁₀ hydrocarbon-based chain, which can form one or more 4- to 8-membered carbon-based rings, and which may be saturated or unsaturated, and the carbon atoms of which may be, independently of each other, substituted with one or more halogen atoms or hydroxyl, amino, monosubstituted or disubstituted amino, C₁-C₄ alkoxy, C₁-C₄ thioether, carboxyl, sulphonic or thiol radicals;
hydroxyl or amino radicals, the amine possibly being substituted with a linear or branched C₁-C₄ hydrocarbon-based chain, which can form one or more 5- or 6-membered carbon-based rings, and which may be saturated or unsaturated, the carbon atoms may be, independently of each other, substituted with one or more halogen atoms or hydroxyl or amino radicals.

When W₁ is a pyrazolo[1,5-b]pyridine radical, the radicals R₆, R₇, R₈, R₉ and R₁₅ are preferably chosen from a hydrogen atom, a linear or branched C₁-C₄ hydrocarbon-based chain which may be saturated or unsaturated, the carbon atoms may be, independently of each other, substituted with one or more halogen atoms or hydroxyl or amino radicals.

W₁ may also be a pyrazolo[1,5-a]pyrimidine radical in which R₇ and R₉ are chosen from a hydrogen atom, a linear or branched C₁-C₆ alkyl radical; a C₁-C₆ monohydroxyalkyl radical; a C₂-C₆ polyhydroxyalkyl radical; a C₁-C₆ aminoalkyl radical or a C₁-C₆ aminoalkyl radical in which the amine is mono- or disubstituted with a (C₁-C₆)alkyl or (C₁-C₆)alkylcarbonyl radical, a hydroxyl or amino radical, the amino possibly being substituted with a linear or branched C₁-C₁₀ hydrocarbon-based chain, which can form one or more 5- or 6-membered carbon-based rings which may be saturated or unsaturated, the carbon atoms may be, independently of each other, substituted with one or more halogen atoms or hydroxyl or amino radicals; R₆ and R₈ are chosen from a hydrogen atom, a linear or branched C₁-C₆ alkyl radical; a C₁-C₆ monohydroxyalkyl radical; a C₂-C₆ polyhydroxyalkyl radical; a C₁-C₆ aminoalkyl radical or a C₁-C₆ aminoalkyl radial in which the amine is mono- or disubstituted with a (C₁-C₆)alkyl or (C₁-C₆)alkylcarbonyl radical. In this case, R₇ and R₉ are preferably chosen from a hydrogen atom; a linear or branched C₁-C₄ alkyl radical; a C₁-C₄ monohydroxyalkyl radical; a C₂-C₄ polyhydroxyalkyl radical; a C₁-C₄ aminoalkyl radical or a C₁-C₄ aminoalkyl in which the amine is mono- or disubstituted with a (C₁-C₂)alkyl radical, a hydroxyl or amino radical, the amino possibly being substituted with a linear or branched C₁-C₄ hydrocarbon-based chain, the carbon atoms may be, independently of each other, substituted with one or more hydroxyl or amino radicals, and R₆ and R₈ are preferably chosen from a hydrogen atom, a linear or branched C₁-C₄ alkyl radical; a C₁-C₄ monohydroxyalkyl radical; a C₂-C₄ polyhydroxyalkyl radical; a C₁-C₄ aminoalkyl radical or a C₁-C₄ aminoalkyl radical in which the amine is mono- or disubstituted with a (C₁-C₂)alkyl radical; a C₁-C₂ alkoxy radical.

According to one particularly preferred embodiment, W₁ is a pyrazolopyridine or pyrazolopyrimidine derivative in which R₆, R₇, R₈ and R₉ are preferably chosen from a hydrogen atom; a C₁-C₄ alkyl radical; an amino radical; a C₁-C₄ mono- or dialkylamino radical; a C₁-C₄ hydroxyalkyl radical or a C₁-C₂ alkoxy radical.

The compound of formula (I) may be represented by the following formula 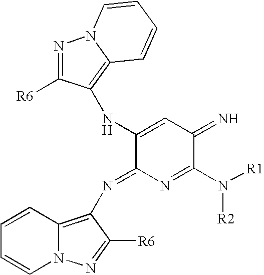
in which R1, R2 and R6 are as defined above.

By way of example, the compounds of formula (I) may be cited

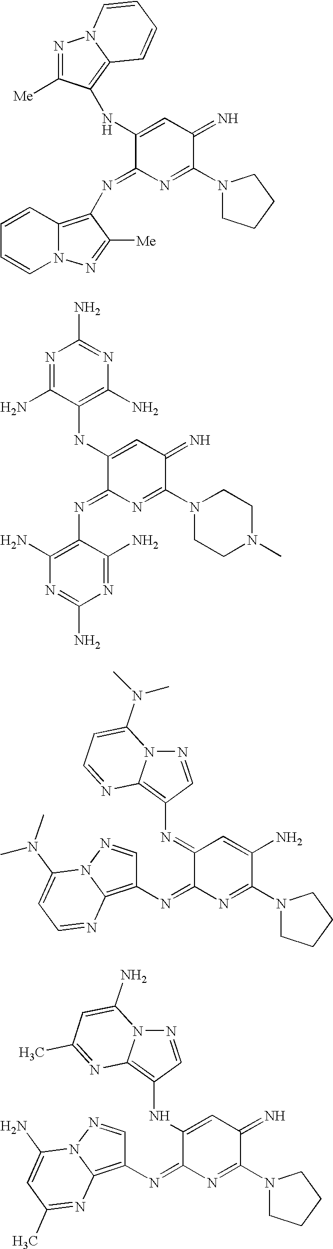

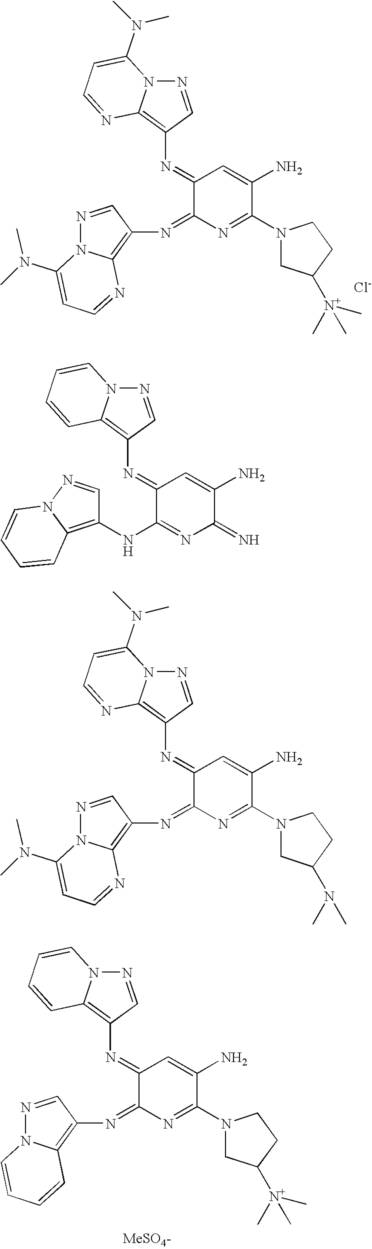

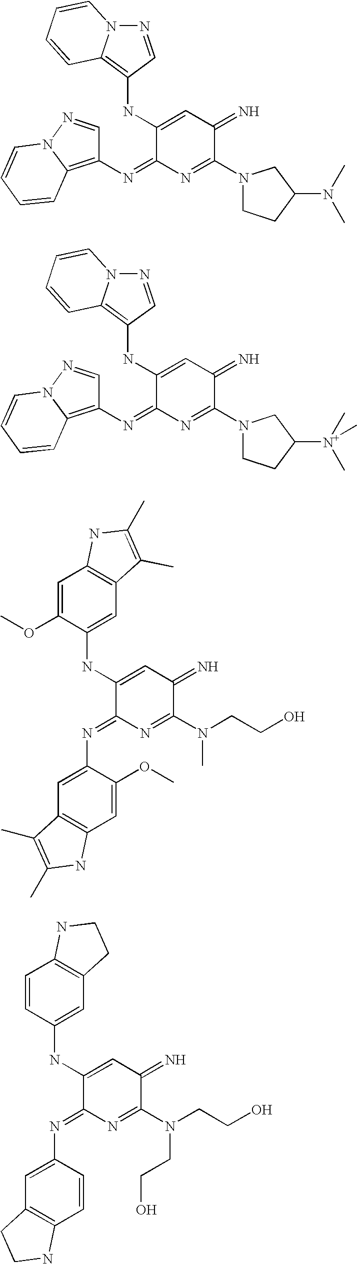

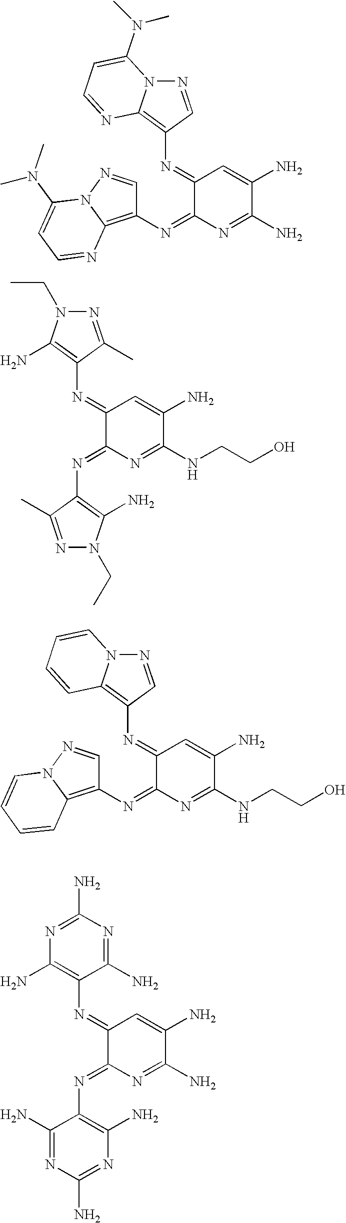

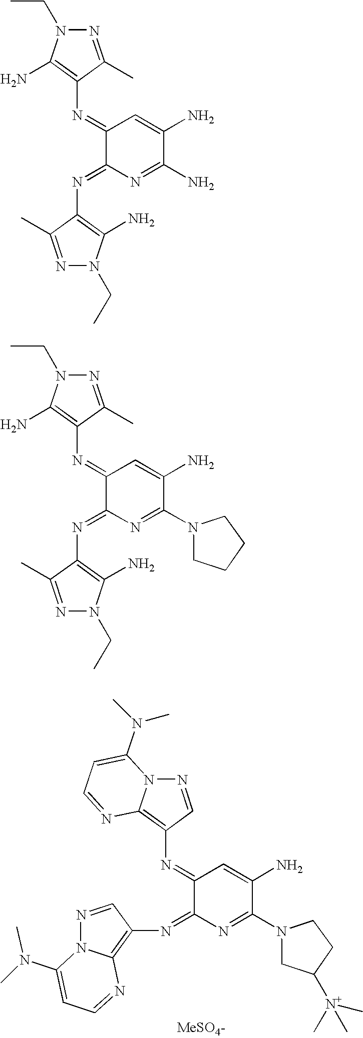

The composition of the invention preferably contains an amount of compound of formula (I) of between 0.01% and 10% by weight and preferably between 0.1% to 5% by weight.

The dye composition in accordance with the invention may also contain direct dyes other than the compounds of formula (I). These direct dyes that are useful according to the invention are chosen, for example, from neutral, acidic or cationic nitrobenzene direct dyes, neutral, acidic or cationic azo direct dyes, quinone direct dyes and in particular neutral, acidic or cationic anthraquinone direct dyes, azine direct dyes, triarylmethane direct dyes, indoamine direct dyes and natural direct dyes.

Among the benzenic direct dyes that may be used according to the invention, mention may be made, in a nonlimiting manner, of the following compounds:
1,4-diamino-2-nitrobenzene
1-amino-2-nitro-4-β-hydroxyethylaminobenzene
1-amino-2-nitro-4-bis(β-hydroxyethyl)aminobenzene
1,4-bis(β-hydroxyethylamino)-2-nitrobenzene
1-β-hydroxyethylamino-2-nitro-4-bis(β-hydroxyethylamino)benzene
1-β-hydroxyethylamino-2-nitro-4-aminobenzene
1-β-hydroxyethylamino-2-nitro-4-(ethyl)(β-hydroxyethyl)aminobenzene
1-amino-3-methyl-4-β-hydroxyethylamino-6-nitrobenzene
1-amino-2-nitro-4-β-hydroxyethylamino-5-chlorobenzene
1,2-diamino-4-nitrobenzene
1-amino-2-β-hydroxyethylamino-5-nitrobenzene
1,2-bis(β-hydroxyethylamino)-4-nitrobenzene
1-amino-2-tris(hydroxymethyl)methylamino-5-nitrobenzene
1-hydroxy-2-amino-5-nitrobenzene
1-hydroxy-2-amino-4-nitrobenzene
1-hydroxy-3-nitro-4-aminobenzene
1-hydroxy-2-amino-4,6-dinitrobenzene
1-β-hydroxyethyloxy-2-β-hydroxyethylamino-5-nitrobenzene
1-methoxy-2-β-hydroxyethylamino-5-nitrobenzene
1-β-hydroxyethyloxy-3-methylamino-4-nitrobenzene
1-β,γ-dihydroxypropyloxy-3-methylamino-4-nitrobenzene
1-β-hydroxyethylamino-4-β,γ-dihydroxypropyloxy-2-nitrobenzene
1-β,γ-dihydroxypropylamino-4-trifluoromethyl-2-nitrobenzene
1-β-hydroxyethylamino-4-trifluoromethyl-2-nitrobenzene
1-β-hydroxyethylamino-3-methyl-2-nitrobenzene
1-β-aminoethylamino-5-methoxy-2-nitrobenzene
1-hydroxy-2-chloro-6-ethylamino-4-nitrobenzene
1-hydroxy-2-chloro-6-amino-4-nitrobenzene
1-hydroxy-6-bis(β-hydroxyethyl)amino-3-nitrobenzene
1-β-hydroxyethylamino-2-nitrobenzene
1-hydroxy-4-β-hydroxyethylamino-3-nitrobenzene.

Among the azo direct dyes that may be used according to the invention, mention may be made of the cationic azo dyes described in patent applications WO 95/15144, WO-95/01772 and EP-714 954, the content of which forms an integral part of the invention.

Among these compounds that may be mentioned most particularly are the following dyes:
1,3-dimethyl-2-[[4-(dimethylamino)phenyl]azo]-1H-imidazolium chloride,
1,3-dimethyl-2-[(4-aminophenyl)azo]-1H-imidazolium chloride,
1-methyl-4-[(methylphenylhydrazono)methyl]pyridinium methyl sulphate.

Among the azo direct dyes that may also be mentioned are the following dyes, described in the Color Index International 3rd. edition:
Disperse Red 17
Acid Yellow 9
Acid Black 1
Basic Red 22
Basic Red 76
Basic Yellow 57
Basic Brown 16
Acid Yellow 36
Acid Orange 7
Acid Red 33
Acid Red 35
Basic Brown 17
Acid Yellow 23
Acid Orange 24
Disperse Black 9.

Mention may also be made of 1-(4′-aminodiphenylazo)-2-methyl-4-bis(β-hydroxyethyl)aminobenzene and 4-hydroxy-3-(2-methoxyphenylazo)-1-naphthalenesulphonic acid.

Among the quinone direct dyes that may be mentioned are the following dyes:
Disperse Red 15
Solvent Violet 13
Acid Violet 43
Disperse Violet 1
Disperse Violet 4
Disperse Blue 1
Disperse Violet 8
Disperse Blue 3
Disperse Red 11
Acid Blue 62
Disperse Blue 7
Basic Blue 22
Disperse Violet 15
Basic Blue 99
   and also the following compounds:
1-N-Methylmorpholiniumpropylamino-4-hydroxyanthraquinone
1-Aminopropylamino-4-methylaminoanthraquinone
1-Aminopropylaminoanthraquinone
5-β-Hydroxyethyl-1,4-diaminoanthraquinone
2-Aminoethylaminoanthraquinone
1,4-Bis(β,γ-dihydroxypropylamino)anthraquinone.

Among the azine dyes that may be mentioned are the following compounds:
Basic Blue 17
Basic Red 2.

Among the triarylmethane dyes, mention may be made of the following compounds:
Basic Green 1
Acid Blue 9
Basic Violet 3
Basic Violet 14
Basic Blue 7
Acid Violet 49
Basic Blue 26
Acid Blue 7.

Among the indoamine dyes, mention may be made of the following compounds:
2-β-Hydroxyethylamino-5-[bis(β-4′-hydroxyethyl)amino]anilino-1,4-benzoquinone
2-β-Hydroxyethylamino-5-(2′-methoxy-4′-amino)anilino-1,4-benzoquinone
3-N-(2′-Chloro-4′-hydroxy)phenylacetylamino-6-methoxy-1,4-benzoquinone imine
3-N-(3′-Chloro-4′-methylamino)phenylureido-6-methyl-1,4-benzoquinone imine
3-[4′-N-(Ethyl,carbamylmethyl)amino]phenylureido-6-methyl-1,4-benzoquinone imine.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin and apigenidin. It is also possible to use extracts or decoctions containing these natural dyes, and especially henna-based poultices or extracts.

The direct dye(s) preferably represent(s) from 0.001% to 20% by weight approximately relative to the total weight of the ready-to-use composition, and even more preferably from 0.005% to 10% by weight approximately.

The composition of the present invention may also contain oxidation bases and couplers conventionally used for oxidation dyeing.

Examples that may be mentioned include para-phenylenediamines, bis(phenyl)alkylenediamines, para-aminophenols, ortho-aminophenols and heterocyclic bases, and the addition salts thereof.

The couplers are, for example, meta-phenylenediamine couplers, meta-aminophenol couplers, meta-diphenol couplers, naphthalene-based couplers and heterocyclic couplers, and the addition salts thereof.

When they are present, the bases and couplers are each generally present in an amount of between 0.001% and 10% by weight approximately and preferably between 0.005% and 6% by weight relative to the total weight of the dye composition.

The medium that is suitable for dyeing, also known as the dye support, generally consists of water or of a mixture of water and of at least one organic solvent to dissolve the compounds that would not be sufficiently water-soluble. Examples of organic solvents that may be mentioned include C₁-C₄ lower alkanols, such as ethanol and isopropanol; polyols and polyol ethers, for instance 2-butoxyethanol, propylene glycol, propylene glycol monomethyl ether and diethylene glycol monoethyl ether and monomethyl ether, and also aromatic alcohols, for instance benzyl alcohol or phenoxyethanol, and mixtures thereof.

For dyeing human keratin fibres, the dyeing medium is a suitable cosmetic medium.

The solvents may be present in proportions preferably of between 1% and 40% by weight approximately and even more preferably between 5% and 30% by weight approximately relative to the total weight of the dye composition.

The dye composition in accordance with the invention may also contain various adjuvants conventionally used in compositions for dyeing the hair, such as anionic, cationic, nonionic, amphoteric or zwitterionic surfactants or mixtures thereof, anionic, cationic, nonionic, amphoteric or zwitterionic polymers or mixtures thereof, mineral or organic thickeners, and in particular anionic, cationic, nonionic and amphoteric polymeric associative thickeners, antioxidants, penetrating agents, sequestering agents, fragrances, buffers, dispersants, conditioners, for instance volatile or non-volatile, modified or unmodified silicones, film-forming agents, ceramides, preserving agents and opacifiers.

The above adjuvants are generally present in an amount for each one of between 0.01% and 20% by weight relative to the weight of the composition.

Needless to say, a person skilled in the art will take care to select this or these optional additional compound(s) such that the advantageous properties intrinsically associated with the oxidation dye composition in accordance with the invention are not, or are not substantially, adversely affected by the envisaged addition(s).

The pH of the dye composition in accordance with the invention is generally between 3 and 12 approximately and preferably between 5 and 11 approximately.

It may be adjusted to the desired value by means of acidifying or basifying agents usually used in the dyeing of keratin fibres or using standard buffer systems.

Among the acidifying agents that may be mentioned, for example, are mineral or organic acids, for instance hydrochloric acid, orthophosphoric acid, sulphuric acid, carboxylic acids, for instance acetic acid, tartaric acid, citric acid or lactic acid, and sulphonic acids.

Among the basifying agents that may be mentioned, for example, are aqueous ammonia, alkali metal carbonates, alkanolamines such as mono-, di- or triethanolamine and also derivatives thereof, sodium hydroxide, potassium hydroxide and the compounds of formula (III) below: 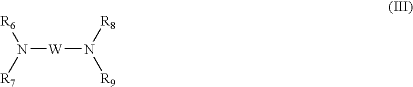
in which W is a propylene residue optionally substituted with a hydroxyl group or a C₁-C₄ alkyl radical; R₆, R₇, R₈ and R₉, which may be identical or different, represent a hydrogen atom or a C₁-C₄ alkyl or C₁-C₄ hydroxyalkyl radical.

The dye composition according to the invention may be in various forms, such as in the form of liquids, creams or gels, or in any other form that it suitable for dyeing keratin fibres, and especially human hair.

A subject of the invention is also a process of direct dyeing which comprises the application of a dye composition containing a dye of formula (I) as defined above to keratin fibres. After an action time, the keratin fibres are rinsed, allowing the coloured fibres to show. The action time is generally between 3 and 50 minutes approximately and preferably 5 to 30 minutes approximately.

When the dye composition comprises an oxidation base and/or a coupler, the dye composition may then contain an oxidizing agent. The oxidizing agents conventionally used for the oxidation dyeing of keratin fibres are, for example, hydrogen peroxide, urea peroxide, alkali metal bromates, persalts such as perborates and persulphates, peracids and oxidase enzymes, among which mention may be made of peroxidases, 2-electron oxidoreductases such as uricases, and 4-electron oxygenases, for instance laccases. Hydrogen peroxide is particularly preferred.

The oxidizing agent may be added to the composition just at the time of use, or it may be used starting with an oxidizing composition containing it, which is applied simultaneously or sequentially to the composition of the invention. The oxidizing composition may also contain various adjuvants conventionally used in compositions for dyeing the hair and as defined above.

The pH of the oxidizing composition containing the oxidizing agent is such that, after mixing with the dye composition, the pH of the resulting composition applied to the keratin fibres preferably ranges between 3 and 12 approximately and even more preferably between 5 and 11. It may be adjusted to the desired value using acidifying or basifying agents usually used in the dyeing of keratin fibres and as defined above.

The composition that is finally applied to the keratin fibres may be in various forms, such as in the form of liquids, creams or gels or in any other form that is suitable for dyeing keratin fibres, and especially human hair.

The compounds of the invention of formula (I) may be prepared in the following way:

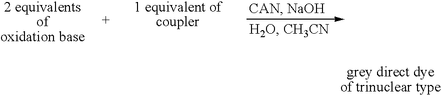

The term “oxidation base” means the heterocyclic oxidation bases conventionally used in dyeing, which can, after reaction with the coupler, form a radical W₁. The coupler used is a 2,3-diaminopyridine coupler substituted in position 6 with a leaving group such as an alkoxy, a halogen atom, an aryloxy, —OSO₂R, a radical —OCOR, or a C₁-C₃ trifluoroalkoxy radical, with R representing an alkyl radical.

Heterocyclic oxidation bases that may be mentioned include pyridine derivatives, pyrimidine derivatives and pyrazole derivatives.

According to one particular embodiment, the base and the coupler are mixed with a solvent, for example acetonitrile. A base, for example sodium hydroxide, and CAN are added to this mixture. After (a period of) stirring, water is added to the reaction medium. The aqueous phase is extracted, for example with ethyl acetate. The organic phase is removed. The aqueous phase is re-extracted, for example with butanol. The butanol phase is then dried, filtered and then concentrated and purified.

The examples that follow serve to illustrate the invention without, however, being limiting in nature.

### EXAMPLES

### Example 1

### Preparation of a Compound of Formula

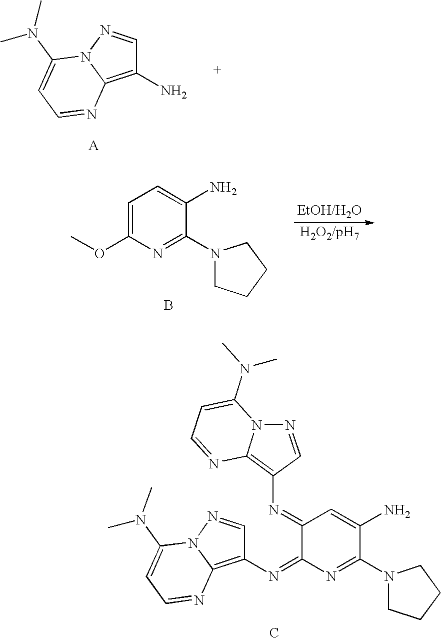

3-Amino-7-diethylaminopyrazolopyrimidine (A: 0.440 g), 2-pyrrolidino-3-amino-6-methoxypyridine (B: 0.386 g), ethanol/water ((60/40), 40 g), H₂O₂ (20 vol., 50 g) and a buffer at pH 7 (10 g of buffer: KH₂PO₄ 13.6 g; K₂HPO₄ 26.1 g and water qs 100 ml) are mixed together and stirred at room temperature for 1 hour. The reaction mixture is then precipitated and the dye obtained (C) is purified by column chromatography.

NMR (¹H CD₃OD, 400 MHz), 3.18 ppm (s, 12H); 3.97 ppm (s, 4H); 6.21 ppm (s, 1H); 6.27 ppm (s, 1H); 6.62 ppm (d, 1H); 6.71 ppm (d, 1H); 7.95 ppm (s, 1H); 8.39 ppm (d, 1H); 8.41 ppm (d, 1H); 8.83 ppm (s, 1H)

ESI+/MS M=511 (2 exchangeable protons)

MS/MS m/z=485 [M+H−HCN]+; m/z=350 [M+H−C₈H₁₀N₄]+ and m/z=323 [M+H−HCN−C₈H₁₀N₄]+ and m/z=176 [C₈N₅H₁₁−H]+

### Example 2

### Preparation of a Compound of Formula

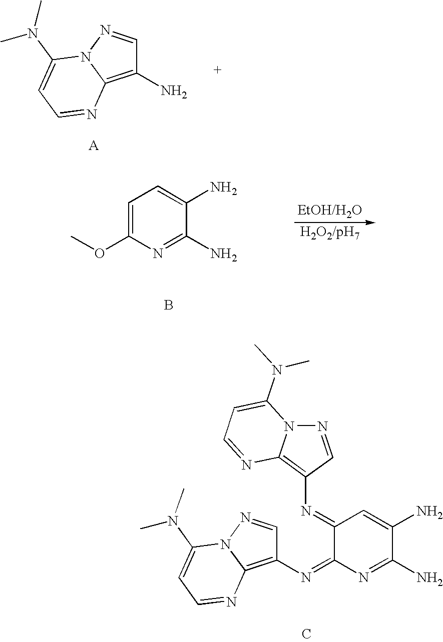

0.327 g of 3-amino-7-diethylaminopyrazolopyrimidine (A: 1.31 mmol; 2 eq.) and 0.138 g of 2,3-diamino-6-methoxypyridine (B: 0.65 mmol; 1 eq.) are dissolved in 80 ml of absolute ethanol. 8 ml of 6% aqueous hydrogen peroxide solution are then added and stirring is continued at room temperature. After one week, the mixture is filtered and the black dye (C) is recovered.

NMR (¹H, DMSO-d6, 400 MHz): 3.14 ppm (s, 6H); 3.20 ppm (s, 6H); 6.34 ppm (s, 1H); 6.70-6.66 (2d, 2H); 8.13 ppm (s, 1H); 8.68-8.63 ppm (2d, 2H); 9.11 (s, 1H); 10.21 (broad s, 1H exchangeable).

Mass (ES+): m/z: 458 (MH^{+.})

### Example 3

### Preparation of a Compound of Formula

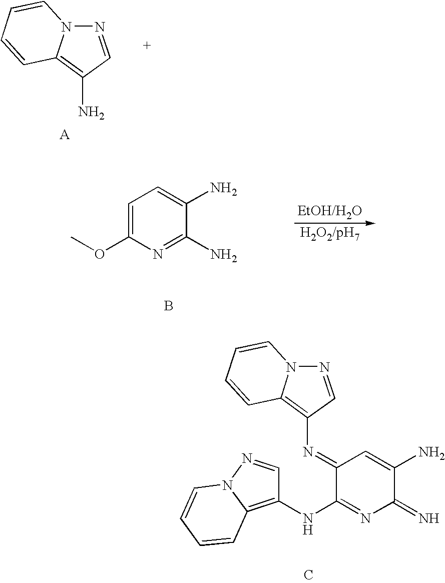

0.222 g of 3-aminopyrazolopyridine (A: 1.31 mmol; 2 eq.) and 0.138 g of 2,3-diamino-6-methoxypyridine (B: 0.65 mmol; 1 eq.) are dissolved in 80 ml of absolute ethanol. 8 ml of 6% aqueous hydrogen peroxide solution are then added and stirring is continued at room temperature. After one week, the mixture is filtered and the solid dye (C) is recovered.

NMR (¹H DMSO-d6, 400 MHz): 5.98 ppm (s, 1H); 7.12-7.04 (2dd, 2H); 7.37 ppm (dd, 1H); 7.52-7.49 ppm (dd, 1H); 7.70-7.68 ppm (d, 1H); 8.29 ppm (s, 1H); 8.52-8.50 ppm (d, 1H); 8.80-8.75 ppm (2dd, 2H); 9.22 ppm (s, 1H); 9.54 ppm (broad s, 2H exchangeable); 10.66 (broad s, 1H exchangeable).

Mass (ES+): m/z: 370 (MH^{+.})

### Example 4

### Preparation of a Compound of Formula

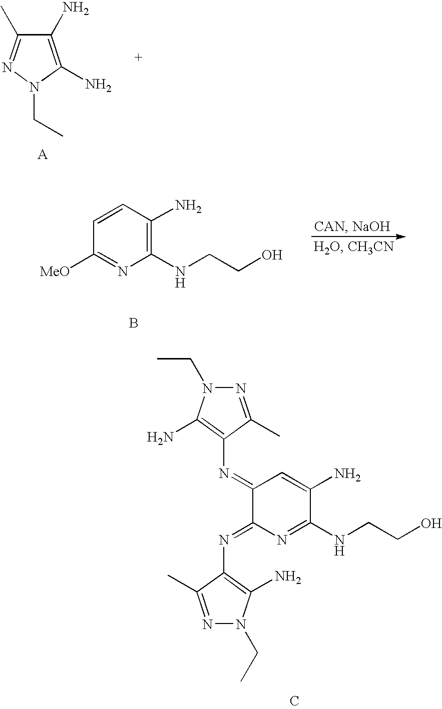

0.097 g of A (0.45 mmol; 2 eq.) and 0.050 g of B (0.22 mmol; 1 eq.) are placed in 10 ml of acetonitrile. 338 mg of 35% sodium hydroxide diluted in 10 ml of water are then added, immediately followed by 499 mg of CAN. The solution turns a grey-blue colour within a few minutes. After stirring for 4 hours, 100 ml of water are added. The aqueous phase is extracted with ethyl acetate. The organic phase is removed. The aqueous phase is re-extracted with butanol. The butanol phase is then dried over sodium sulphate, filtered and then concentrated. The product is taken up in ethanol (2 ml) and then reprecipitated in diisopropyl ether. A black powder is obtained (C: m=0.081 g).

Mass (ES+): m/z: 428 (MH^{+.})

### Example 5

### Preparation of a Compound of Formula

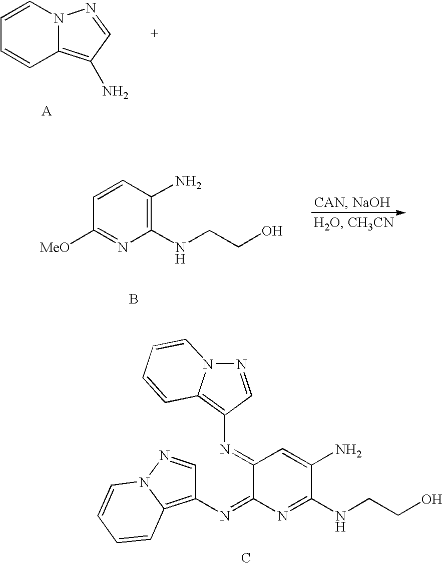

0.169 g of A (0.45 mmol; 2 eq.) and 0.050 g of B (0.22 mmol; 1 eq.) are placed in 10 ml of acetonitrile. 338 mg of 35% sodium hydroxide diluted in 10 ml of water are then added, immediately followed by 499 mg of CAN (Cerium (IV) Ammonium Nitrate: (NH₄)₂Ce(NO₃)₆).

The solution turns a grey colour within a few minutes. After stirring for 4 hours, 100 ml of water are added. The aqueous phase is extracted with ethyl acetate. The organic phase is removed. The aqueous phase is re-extracted with butanol. The butanol phase is then dried over sodium sulphate, filtered and then concentrated. The product is taken up in ethanol (2 ml) and then reprecipitated in diisopropyl ether. A black powder is obtained (C).

Mass (ES+): m/z: 414 (MH^{+.})

### Example 6

### Preparation of a Compound of Formula

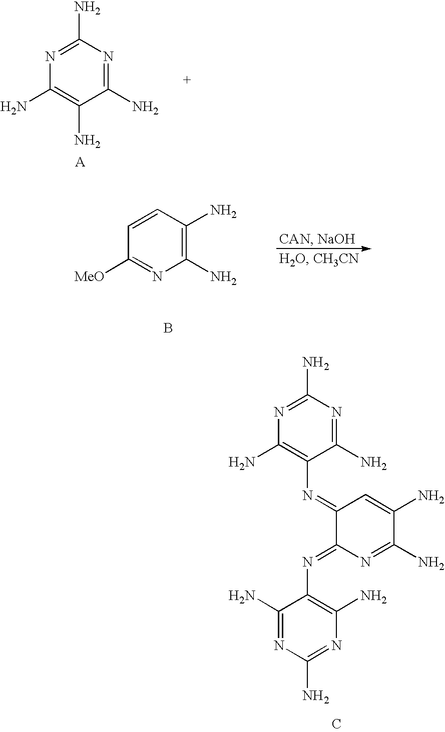

0.238 g of A (0.45 mmol; 2 eq.) and 0.050 g of B (0.22 mmol; 1 eq.) are placed in 10 ml of acetonitrile. 338 mg of 35% sodium hydroxide diluted in 10 ml of water are then added, immediately followed by 499 mg of CAN. The solution turns a grey colour within a few minutes. After stirring for 4 hours, 100 ml of water are added. The aqueous phase is extracted with ethyl acetate. The organic phase is removed. The aqueous phase is re-extracted with butanol. The butanol phase is then dried over sodium sulphate, filtered and then concentrated. The product is taken up in ethanol (2 ml) and then reprecipitated in diisopropyl ether. A black powder is obtained (C).

Mass (ES+): m/z: 384 (MH^{+.})

### Example 7

### Preparation of a Compound of Formula

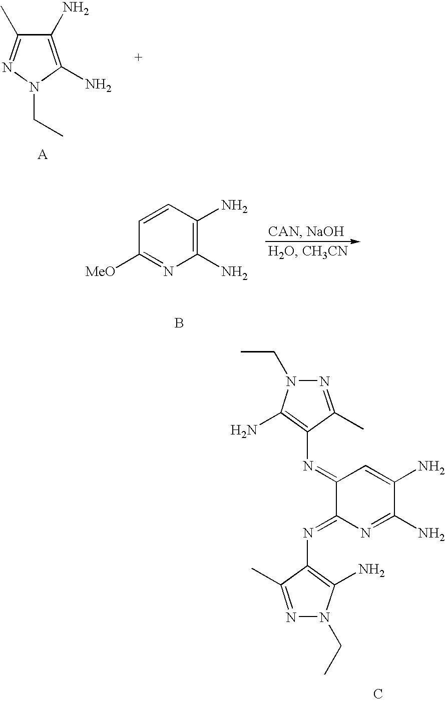

0.097 g of A (0.45 mmol; 2 eq.) and 0.050 g of B (0.22 mmol; 1 eq.) are placed in 10 ml of acetonitrile. 338 mg of 35% sodium hydroxide diluted in 10 ml of water are then added, immediately followed by 499 mg of CAN. The solution turns a grey-blue colour within a few minutes. After stirring for 4 hours, 100 ml of water are added. The aqueous phase is extracted with ethyl acetate. The organic phase is removed. The aqueous phase is re-extracted with butanol. The butanol phase is then dried over sodium sulphate, filtered and then concentrated. The product is taken up in ethanol (2 ml) and then reprecipitated in diisopropyl ether. A black powder is obtained (C).

Mass (ES+): m/z: 384 (MH^{+.})

### Example 8

### Preparation of a Compound of Formula:

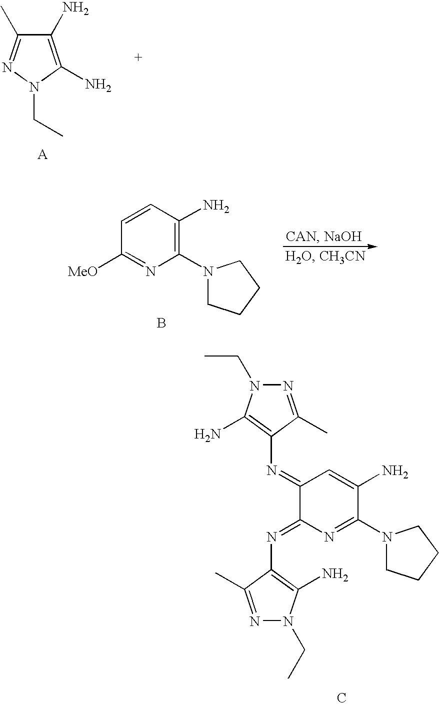

0.097 g of A (0.45 mmol; 2 eq.) and 0.058 g of B (0.22 mmol; 1 eq.) are placed in 10 ml of acetonitrile. 338 mg of 35% sodium hydroxide diluted in 10 ml of water are then added, immediately followed by 499 mg of CAN. The solution turns a grey-blue colour within a few minutes. After stirring for 4 hours, 100 ml of water are added. The aqueous phase is extracted with ethyl acetate. The organic phase is removed. The aqueous phase is re-extracted with butanol. The butanol phase is then dried over sodium sulphate, filtered and then concentrated. The product is taken up in ethanol (2 ml) and then reprecipitated in diisopropyl ether. A black powder is obtained (C).

Mass (ES+): m/z: 438 (MH^{+.})

By following the synthetic process described above, the compounds below may be obtained according to the indicated synthetic scheme: 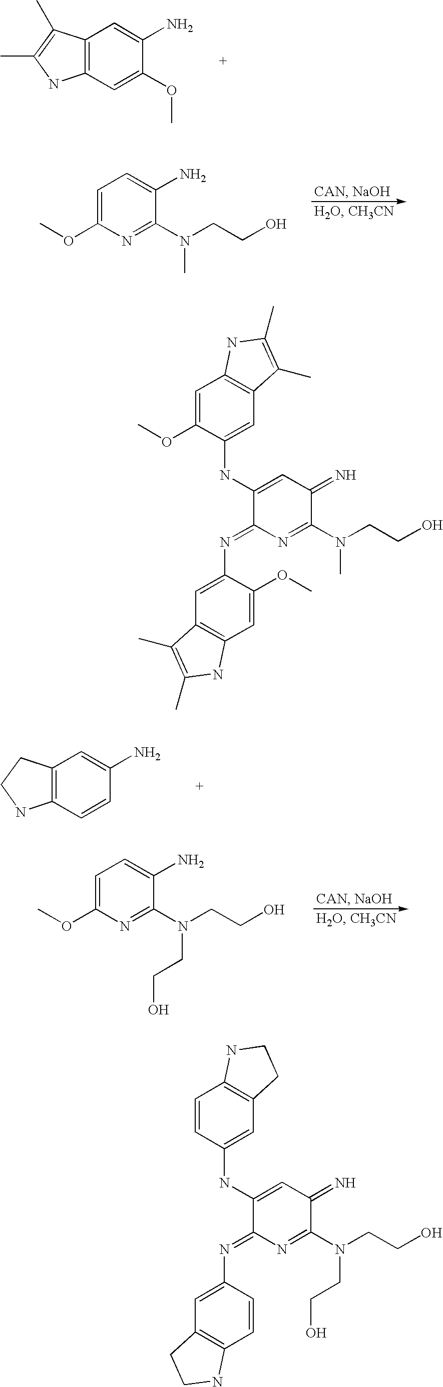 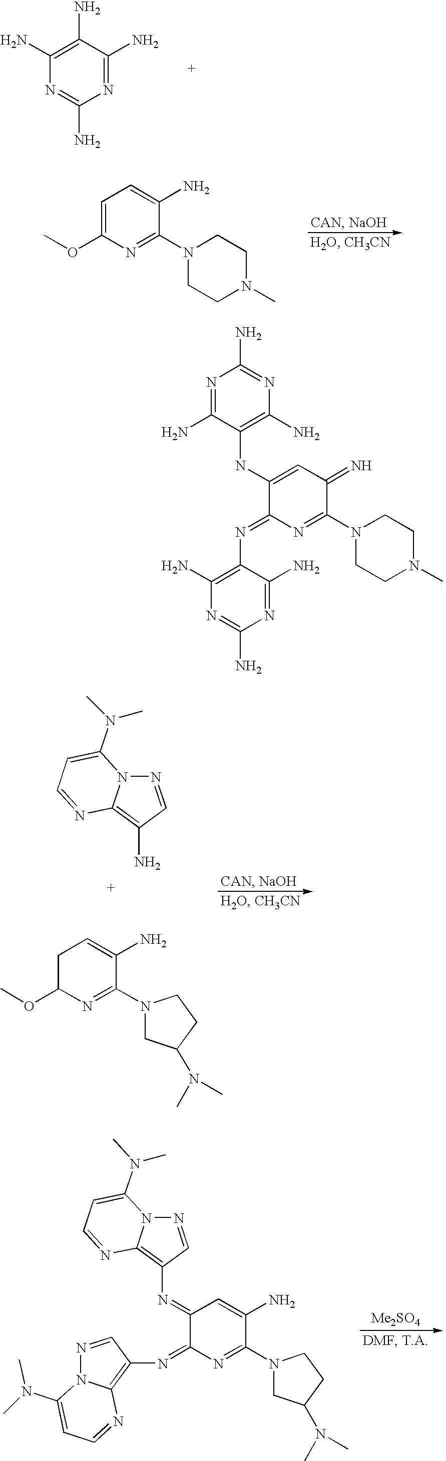 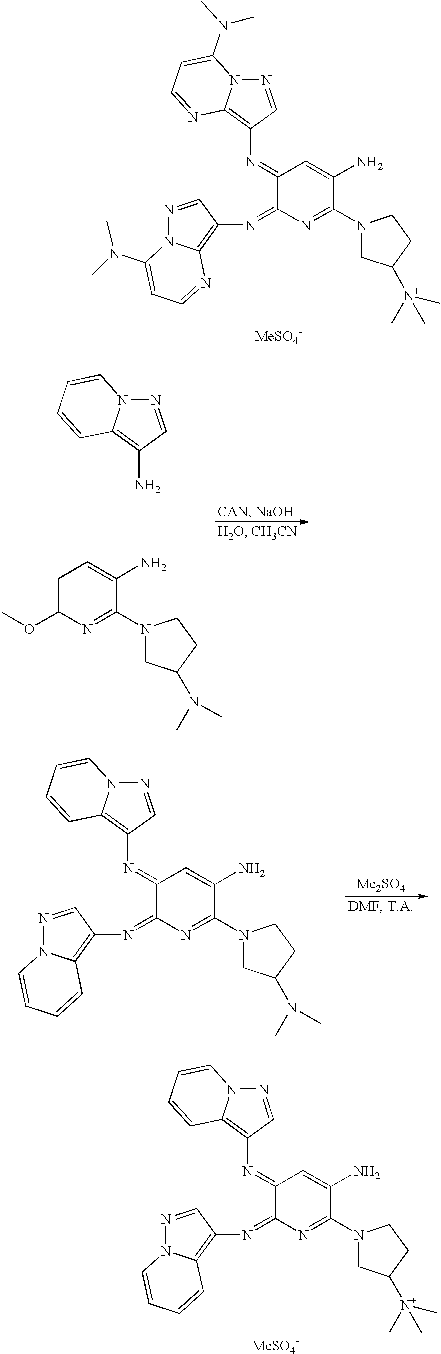
Evaluation in the Dyeing of Keratin Fibres

Each of the dyes of the preceding examples is mixed in the medium below to form a dye composition (0.5% by weight of dye). Keratin fibres are then added to the composition thus obtained (composition/fibre ratio=10/1). After 30 minutes, the hair is rinsed with water and then dried. A black coloration of the hair is thus obtained.

Dyeing Medium

0.768% hydroxyethylcellulose
0.064% parabens
10% decyl glucoside
8% benzyl alcohol
12% propylene glycol
10% pH 7 buffer
water qs 100%

1-46. (Canceled)

47. A dye composition comprising, in a suitable medium, at least one compound of formula (I), or an addition salt thereof: 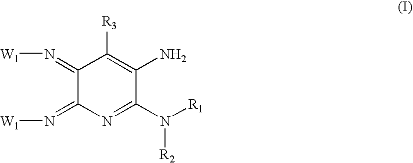
wherein
R₁ and R₂, which are independent of each other, are chosen from:
hydrogen atoms,
linear and branched, unsaturated and saturated C₁-C₁₀ hydrocarbon-based chains, which optionally form at least one 4- to 8-membered carbon-based ring, wherein at least one carbon atom of the carbon-based chain, independently of the other carbon atoms, is optionally replaced with an entity chosen from oxygen, nitrogen and sulphur atoms and SO₂ groups, and optionally substituted with at least one entity chosen from halogen atoms and hydroxyl, amino, C₁-C₂ (di)alkylamino, C₁-C₂ alkoxy, carboxyl, sulphonic, and thiol radicals; with the proviso that R₁ and R₂ do not comprise a peroxide bond, or a diazo or nitroso radical, and R₁ and R₂ are not directly linked to the nitrogen atom via an oxygen, sulphur or nitrogen atom or a SO₂ group, and
an onium radical Z,
R₁ and R₂ form, with the nitrogen atom to which they are attached, a ring of formula (II): 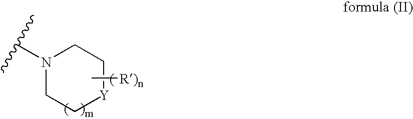
wherein
R′ is chosen from:
a hydrogen atom;
a halogen atom;
a C₁-C₄ alkyl radical optionally substituted with at least one radical chosen from hydroxyl, carboxyl, C₁-C₄ alkoxycarbonyl, (C₁-C₄)alkylamido((C₁-C₄)alkylCONH—), (C₁-C₄)alkylcarbamoyl ((C₁-C₄)alkylNHCO—), (C₁-C₄)alkylsulphonyl ((C₁-C₄)alkylSO₂—), C₁-C₄ alkoxy, (C₁-C₄)alkylsulphonamido ((C₁-C₄)alkylSO₂NH—), (C₁-C₄)alkylsulphamoyl ((C₁-C₄)alkylNHSO₂—), and onium Z radicals;
a NR′₃R′₄ radical;
a carboxyl radical;
a C₁-C₄ alkoxycarbonyl radical;
a (C₁-C₄)alkylamido radical ((C₁-C₄)alkylCONH—);
a (C₁-C₄)alkylsulphonyl radical (alkylSO₂—);
an alkylsulphonamido radical ((C₁-C₄)alkylSO₂NH—);
a hydroxyl radical;
a C₁-C₄ alkoxy radical;
a C₂-C₄ hydroxyalkoxy radical;
a (C₁-C₄)alkylcarbamoyl radical ((C₁-C₄)alkylNHCO—);
(C₁-C₄)alkylsulphamoyl ((C₁-C₄)alkyl-NH—SO₂—);
a C₁-C₄ thioether radical;
a sulphonic radical (SO₃H) and the addition salts thereof; and
an onium radical Z,
wherein R′₃ and R′₄, which may be identical or different, are chosen from hydrogen atoms and C₁-C₄ alkyl radicals optionally substituted with at least one radical chosen from hydroxyl, C₁-C₄ alkoxy, amino, mono- and dialkylamino, (C₁-C₄)alkylCO—, (C₁-C₄)alkylNHCO—, and (C₁-C₄)alkylSO₂— radicals,
n is an integer ranging from 1 to 8,
m is an integer ranging from 0 to 3, and
Y is chosen from a oxygen atom, a CR′ radical, a NR′₅ radical, and a NR′₆R′₇ radical wherein
R′₅ is chosen from a hydrogen atom and a linear or branched, saturated or unsaturated C₁-C₁₀ hydrocarbon-based chain, wherein at least one carbon atom of the carbon-based chain, independently of the other carbon atoms, is optionally replaced with an entity chosen from oxygen, nitrogen and sulphur atoms, and SO₂ groups, and optionally substituted with at least one entity chosen from halogen atoms and hydroxyl, amino, carboxyl, sulphonic, and thiol radicals; with the proviso that R′₅ does not comprise a peroxide bond, or a diazo or nitroso radical, and R′₅ is not directly linked to the nitrogen atom via an oxygen, sulphur or nitrogen atom,
R′₆ and R′₇, which are independent of each other, are chosen from linear and branched, saturated and unsaturated C₁-C₁₀ hydrocarbon-based chains, wherein at least one carbon atom of the carbon-based chain, independently from the other carbon atoms, is optionally replaced with an entity chosen from oxygen, nitrogen and sulphur atoms, and SO₂ groups, and optionally substituted with at least one entity chosen from halogen atoms and hydroxyl, amino, carboxyl, sulphonic, and thiol radicals; with the proviso that R′₆ and R′₇ do not comprise a peroxide bond, or a diazo or nitroso radical, and R′₆ and R′₇ are not directly linked to the nitrogen atom via an oxygen, sulphur or nitrogen atom,
R₃ is chosen from:
a hydrogen atom,
a linear or branched, saturated or unsaturated C₁-C₁₀ hydrocarbon-based chain, which optionally forms at least one 4- to 8-membered carbon-based ring, wherein at least one carbon atom of the carbon-based chain, independently of the other carbon atoms, is optionally replaced with an entity chosen from oxygen, nitrogen and sulphur atoms, and SO₂ groups, and optionally substituted with at least one entity chosen from halogen atoms and hydroxyl, amino, C₁-C₂ (di)alkylamino, C₁-C₂ alkoxy, carboxyl, sulphonic, and thiol radicals; with the proviso that R₃ does not comprise a peroxide bond, or a diazo or nitroso radical,
an NR′₁R′₂ radical, wherein R′₁ and R′₂ have the same definitions as R₁ and R₂, respectively,
W₁ is chosen from an aromatic heterocyclic radical chosen from the following radicals: 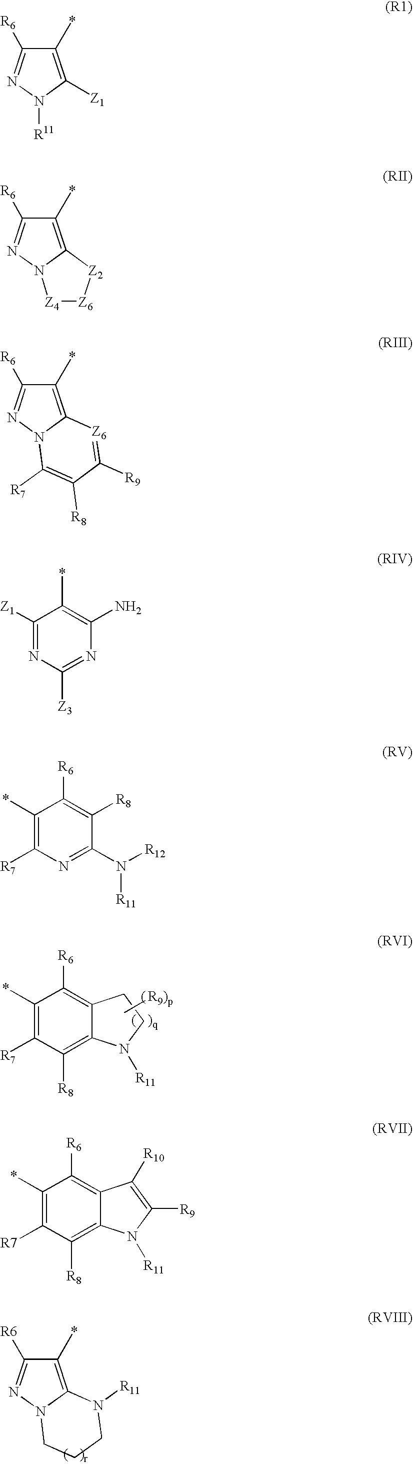
Z₁ and Z₃, which are independent of each other, are chosen from hydrogen atoms, hydroxyl radicals and NR₁₁R₁₂ radicals,
Z₂, Z₄ and Z₆, which are independent of each other, are chosen from nitrogen atoms, CR₁₂ radicals, and NR₁₁ radicals, wherein at least one of Z₂, Z₄ and Z₆ is a CR₁₂ radical and wherein there cannot be more than three contiguous nitrogen atoms,
Z₈ is chosen from a nitrogen atom and a CR₁₅ radical,
R₆, R₇, R₈, R₉, R₁₀, R₁₁, R₁₂ and R₁₅, which are independent of each other, are chosen from:
hydrogen atoms,
linear and branched, saturated and unsaturated C₁-C₁₀ hydrocarbon-based chains, which optionally form at least one 4- to 8-membered carbon-based ring, wherein at least one carbon atom of the carbon-based chain, independently of the the other carbon atoms, is optionally replaced with an entity chosen from oxygen, nitrogen and sulphur atoms, and SO₂ groups, and optionally substituted with at least one entity chosen from halogen atoms and hydroxyl, amnio, carboxyl, sulphonic, and thiol radicals; with the proviso that the radicals R₆ to R₁₂ and R₁₅ do not comprise a peroxide bond, or a diazo or nitroso radical, and the radical R₁₁ is not directly linked to the nitrogen atom via an oxygen, sulphur or nitrogen atom,
p ranges from 4 to 8,
q ranges from 1 to 3, and
r ranges from 0 to 2,
* indicates a point of attachment of W₁ in formula (I).

48. The composition according to claim 47, wherein R₃ is chosen from a hydrogen atom, and a C₁-C₄ alkyl radical optionally substituted with at least one radical chosen from hydroxyl, C₁-C₂ alkoxy, amino, and C₁-C₂ (di)alkylamino radicals.

49. The composition according to claim 47, wherein R₁ and R₂, independently of each other, are chosen from hydrogen atoms and C₁-C₆ alkyl radicals optionally substituted with at least one radical chosen from hydroxyl, alkoxy, amino, and C₁-C₄ (di)alkylamino radicals.

50. The composition according to claim 47, wherein R₁ and R₂ form, with the nitrogen atom to which they are attached, a 5- or 8-membered heterocycle chosen from pyrrolidine, piperidine, homopiperidine, piperazine, homopiperazine, and optionally substituted diazepane heterocycles.

51. The composition according to claim 47, wherein R₁ and R₂ form a heterocycle chosen from pyrrolidine, 3-hydroxypyrrolidine, 3-aminopyrrolidine, 3-acetamidopyrrolidine, 3-(methylsulphonylamino)pyrrolidine, proline, 3-hydroxyproline, piperidine, hydroxypiperidine, homopiperidine, diazepane, N-methylhomopiperazine, N-β-hydroxyethylhomopiperazine, and the addition salts thereof.

52. The composition according to claim 50, wherein R₁ and R₂ form, with the nitrogen atom to which they are attached, an optionally substituted pyrrolidine ring.

53. The composition according to claim 47, wherein the onium radical Z is a radical of formula (III) 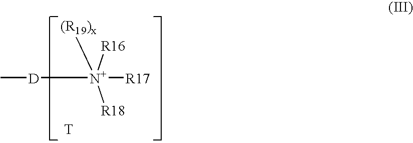
wherein
D is chosen from a covalent bond and a linear and branched C₁-C₁₄ alkylene chain which optionally comprises at least one entity chosen from at least one hetero atom chosen from oxygen, sulphur and nitrogen; SO₂; and at least one ketone function, wherein the chain optionally is substituted with at least one radical chosen from hydroxyl, C₁-C₆ alkoxy, amino, and C₁-C₄ (di)alkylamino radicals,
R₁₆, R₁₇ and R₁₈, which are independent of each other, are chosen from C₁-C₁₅ alkyl radicals; C₁-C₆ monohydroxyalkyl radicals; C₂-C₆ polyhydroxyalkyl radicals; (C₁-C₆)alkoxy(C₁-C₆)alkyl radicals; aryl radicals; benzyl radicals; C₁-C₆ amidoalkyl radicals; tri(C₁-C₆)alkylsilane(C₁-C₆)alkyl radicals; C₁-C₆ aminoalkyl radicals; C₁-C₆ aminoalkyl radicals wherein the amine is mono- or disubstituted with at least one radical chosen from C₁-C₄ alkyl, (C₁-C₆)alkylcarbonyl, amido and (C₁-C₆)alkylsulphonyl radicals; a carbamyl(C₁-C₆)alkyl radical; a (C₁-C₆)alkylcarboxy(C₁-C₆)alkyl radical; a (C₁-C₆)alkylcarbonyl(C₁-C₆)alkyl radical; and a N—(C₁-C₆)alkylcarbamyl(C₁-C₆)alkyl radical;
R₁₆, R₁₇ and R₁₈ together, in pairs, form, with the nitrogen atom to which they are attached, a 4-, 5-, 6- or 7-membered carbon-based saturated ring which optionally comprises at least one hetero atom, wherein the carbon-based ring optionally is substituted with at least one entity chosen from halogen atoms, hydroxyl radicals, C₁-C₆ alkyl radicals, C₁-C₆ monohydroxyalkyl radicals, C₂-C₆ polyhydroxyalkyl radicals, C₁-C₆ alkoxy radicals, tri(C₁-C₆)alkylsilane(C₁-C₆)alkyl radicals, amido radicals, carboxyl radicals, C₁-C₆ alkylcarbonyl radicals, thio radicals, C₁-C₆ thioalkyl radicals, (C₁-C₆)alkylthio radicals, amino radicals, and amino radicals mono- or disubstituted with at least one radical chosen from (C₁-C₆)alkyl, (C₁-C₆)alkylcarbonyl, amido, and (C₁-C₆)alkylsulphonyl radicals;
R₁₉ is chosen from a C₁-C₆ alkyl radical; a C₁-C₆ monohydroxyalkyl radical; a C₂-C₆ polyhydroxyalkyl radical; an aryl radical; a benzyl radical; a C₁-C₆ aminoalkyl radical; a C₁-C₆ aminoalkyl radical wherein the amine is mono- or disubstituted with at least one radicalchosen from (C₁-C₆)alkyl, (C₁-C₆)alkylcarbonyl, amido, and (C₁-C₆)alkylsulphonyl radicals; a carboxy(C₁-C₆)alkyl radical; a carbamyl(C₁-C₆)alkyl radical; a C₁-C₆ trifluoroalkyl radical; a tri(C₁-C₆)alkylsilane(C₁-C₆)alkyl radical; a C₁-C₆ sulphonamidoalkyl radical; a (C₁-C₆)alkylcarboxy(C₁-C₆)alkyl radical; a (C₁-C₆)alkylsulphinyl(C₁-C₆)alkyl radical; a (C₁-C₆)alkylsulphonyl(C₁-C₆)alkyl radical; a (C₁-C₆)alkylcarbonyl(C₁-C₆)alkyl radical; a N—(C₁-C₆)alkylcarbamyl(C₁-C₆)alkyl radical; and a N—(C₁-C₆)alkylsulphonamido(C₁-C₆)alkyl radical;
x is 0 or 1,
when x is equal to 0, then linker arm D is attached to the nitrogen atom bearing the radicals R₁₆ to R₁₈,
when x is equal to 1, then two of the radicals R₁₆ to R₁₈ form, together with the nitrogen atom to which they are attached, a 5-, 6- or 7-membered saturated ring and the linker arm D is linked to a carbon atom of the saturated ring; and
T is a counterion.

54. The composition according to claim 53, wherein when x is equal to 0, R₁₆, R₁₇ and R₁₈, independently of each other, are chosen from C₁-C₆ alkyl radicals, C₁-C₄ monohydroxyalkyl radicals, C₂-C₄ polyhydroxyalkyl radicals, (C₁-C₆)alkoxy(C₁-C₄)alkyl radicals, C₁-C₆ amidoalkyl radicals, and tri(C₁-C₆)alkylsilane(C₁-C₆)alkyl radicals.

55. The composition according to claim 53, wherein when x is equal to 0, R₁₆ and R₁₇ together form a ring chosen from azetidine, pyrrolidine, piperidine, homopiperidine, piperazine, homopiperazine and morpholine rings, and R₁₈ is chosen from a C₁-C₆ alkyl radical; a C₁-C₆ monohydroxyalkyl radical; a C₂-C₆ polyhydroxyalkyl radical; a C₁-C₆ aminoalkyl radical; an aminoalkyl radical wherein the amine is mono- or disubstituted with at least one radical chosen from (C₁-C₄)alkyl, (C₁-C₆)alkylcarbonyl, amido and (C₁-C₆)alkylsulphonyl radicals; a C₁-C₆ carbamylalkyl radical; a tri(C₁-C₆)alkylsilane(C₁-C₆)alkyl radical; a (C₁-C₆)alkylcarboxy(C₁-C₆)alkyl radical; a (C₁-C₆)alkylcarbonyl(C₁-C₆)alkyl radical; and a N—(C₁-C₆)alkylcarbamyl(C₁-C₆)alkyl radical.

56. The composition according to claim 53, wherein when x is equal to 1, R₁₉ is chosen from a C₁-C₆ alkyl radical; a C₁-C₆ monohydroxyalkyl radical; a C₂-C₆ polyhydroxyalkyl radical; a C₁-C₆ aminoalkyl radical; a C₁-C₆ aminoalkyl radical wherein the amine is mono- or disubstituted with at least one radical chosen from (C₁-C₆)alkyl, (C₁-C₆)alkylcarbonyl, amido, and (C₁-C₆)alkylsulphonyl radicals; a C₁-C₆ carbamylalkyl radical; a tri(C₁-C₆)alkylsilane(C₁-C₆)alkyl radical; a (C₁-C₆)alkylcarboxy(C₁-C₆)alkyl radical; a (C₁-C₆)alkylcarbonyl(C₁-C₆)alkyl radical; and a N—(C₁-C₆)alkylcarbamyl(C₁-C₆)alkyl radical, R₁₆ and R₁₇ together form a ring chosen from an azetidine, pyrrolidine, piperidine, homopiperidine, piperazine, homopiperazine, and morpholine rings, and R₁₈ is chosen from a C₁-C₆ alkyl radical; a C₁-C₆ monohydroxyalkyl radical; a C₂-C₆ polyhydroxyalkyl radical; a C₁-C₆ aminoalkyl radical; a C₁-C₆ aminoalkyl radical wherein the amine is mono- or disubstituted with at least one radical chosen from (C₁-C₄)alkyl, (C₁-C₆)alkylcarbonyl, amido, and (C₁-C₆)alkylsulphonyl radicals; a C₁-C₆ carbamylalkyl radical; a tri(C₁-C₆)alkylsilane(C₁-C₆)alkyl radical; a (C₁-C₆)alkylcarboxy(C₁-C₆)alkyl radical; a (C₁-C₆)alkylcarbonyl(C₁-C₆)alkyl radical; and a N—(C₁-C₆)alkylcarbamyl(C₁-C₆)alkyl radical.

57. The composition according to claim 53, wherein x is equal to 0 and R₁₆, R₁₇ and R₁₈ are alkyl radicals.

58. The composition according to claim 53, wherein D is chosen from a covalent bond and a C₁-C₆ alkylene chain, which is optionally substituted.

59. The composition according to claim 47, wherein the onium radical Z is a radical of formula (IV) 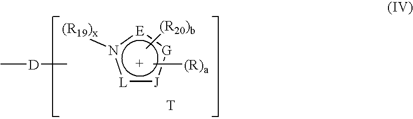
wherein
D is chosen from a covalent bond and a linear and branched C₁-C₁₄ alkylene chain which optionally comprises at least one entity chosen from at least one hetero atom chosen from oxygen, sulphur and nitrogen; SO₂; and at least one ketone function, wherein the chain optionally is substituted with at least one radical chosen from hydroxyl, C₁-C₆ alkoxy, amino, and C₁-C₄ (di)alkylamino radicals,
E, G, J and L, which may be identical or different, are chosen from carbon, oxygen, sulphur, and nitrogen atoms to form a ring chosen from pyrazole, imidazole, triazole, oxazole, isoxazole, thiazole, and isothiazole rings,
a is an integer ranging from 0 to 3;
b is an integer ranging from 0 to 1;
a+b is an integer ranging from 2 to 4,
R, which may be identical or different, is chosen from hydrogen and halogen atoms; a hydroxyl radical; a C₁-C₆ alkyl radical; a C₁-C₆ monohydroxyalkyl radical; a C₂-C₆ polyhydroxyalkyl radical; a C₁-C₆ alkoxy radical; a tri(C₁-C₆)alkylsilane(C₁-C₆)alkyl radical; an amido radical; a carboxyl radical; a C₁-C₆ alkylcarbonyl radical; a thio radical; a C₁-C₆ thioalkyl radical; a (C₁-C₆)alkylthio radical; an amino radical; an amino radical mono- or disubstituted with at least one radical chosen from (C₁-C₆)alkyl, (C₁-C₆)alkylcarbonyl, amido, and (C₁-C₆)alkylsulphonyl radicals; a C₁-C₆ monohydroxyalkyl radical and a C₂-C₆ polyhydroxyalkyl radical; a benzyl radical; and a phenyl radical optionally substituted with at least one radical chosen from methyl, hydroxyl, amino, and methoxy radicals; wherein the radicals R are borne by a carbon atom;
R₂₀ is chosen from a C₁-C₆ alkyl radical, a C₁-C₆ monohydroxyalkyl radical, a C₂-C₆ polyhydroxyalkyl radical, a tri(C₁-C₆)alkylsilane(C₁-C₆)alkyl radical, a (C₁-C₆)alkoxy(C₁-C₆)alkyl radical, a C₁-C₆ carbamylalkyl radical, a (C₁-C₆)alkylcarboxy(C₁-C₆)alkyl radical, and a benzyl radical; wherein the radical R₂₀ is borne by a nitrogen atom,
R₁₉ is chosen from a C₁-C₆ alkyl radical; a C₁-C₆ monohydroxyalkyl radical; a C₂-C₆ polyhydroxyalkyl radical; an aryl radical; a benzyl radical; a C₁-C₆ aminoalkyl radical; a C₁-C₆ aminoalkyl radical wherein the amine is mono- or disubstituted with at least one radical chosen from (C₁-C₆)alkyl, (C₁-C₆)alkylcarbonyl, amido, and (C₁-C₆)alkylsulphonyl radicals; a carboxy(C₁-C₆)alkyl radical; a carbamyl(C₁-C₆)alkyl radical; a C₁-C₆ trifluoroalkyl radical; a tri(C₁-C₆)alkylsilane(C₁-C₆)alkyl radical; a C₁-C₆ sulphonamidoalkyl radical; a (C₁-C₆)alkylcarboxy(C₁-C₆)alkyl radical; a (C₁-C₆)alkylsulphinyl(C₁-C₆)alkyl radical; a (C₁-C₆)alkylsulphonyl(C₁-C₆)alkyl radical; a (C₁-C₆)alkylcarbonyl(C₁-C₆)alkyl radical; a N—(C₁-C₆)alkylcarbamyl(C₁-C₆)alkyl radical; and a N—(C₁-C₆)alkylsulphonamido(C₁-C₆)alkyl radical,
x is equal to 0 or 1,
when x is equal to 0, the linker arm D is attached to the nitrogen atom,
when x is equal to 1, the linker arm D is attached to one ring member chosen from E, G, J, and L when E, G, J or L is chosen from a carbon atom, and
T is a counterion.

60. The composition according to claim 59, wherein the ring members E, G, J and L form a ring chosen from imidazole, pyrazole, oxazole, thiazole, and triazole rings.

61. The composition according to claim 59, wherein x is equal to 0, and D is chosen from a single bond and a C₁-C₄ alkylene chain which is optionally substituted.

62. The composition according to claim 47, wherein the onium radical Z is a radical of formula (V) 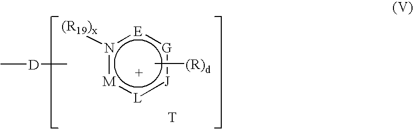
wherein
D is chosen from a covalent bond and a linear and branched C₁-C₁₄ alkylene chain which optionally comprises at least one entity chosen from at least one hetero atom chosen from oxygen, sulphur and nitrogen; SO₂; and at least one ketone function, wherein the chain optionally is substituted with at least one radical chosen from hydroxyl, C₁-C₆ alkoxy, amino, and C₁-C₄ (di)alkylamino radicals,
R, which may be identical or different, is chosen from hydrogen and halogen atoms; a hydroxyl radical; a C₁-C₆ alkyl radical; a C₁-C₆ monohydroxyalkyl radical; a C₂-C₆ polyhydroxyalkyl radical; a C₁-C₆ alkoxy radical; a tri(C₁-C₆)alkylsilane(C₁-C₆)alkyl radical; an amido radical; a carboxyl radical; a C₁-C₆ alkylcarbonyl radical; a thio radical; a C₁-C₆ thioalkyl radical; a (C₁-C₆)alkylthio radical; an amino radical; an amino radical mono- or disubstituted with at least one radical chosen from (C₁-C₆)alkyl, (C₁-C₆)alkylcarbonyl, amido, and (C₁-C₆)alkylsulphonyl radicals; a C₁-C₆ monohydroxyalkyl radical; a C₂-C₆ polyhydroxyalkyl radical; a benzyl radical; and a phenyl radical optionally substituted with at least one radical chosen from methyl, hydroxyl, amino, and methoxy radicals; wherein the radicals R are borne by a carbon atom;
R₁₉ is chosen from a C₁-C₆ alkyl radical; a C₁-C₆ monohydroxyalkyl radical; a C₂-C₆ polyhydroxyalkyl radical; an aryl radical; a benzyl radical; a C₁-C₆ aminoalkyl radical; a C₁-C₆ aminoalkyl radical wherein the amine is mono- or disubstituted with at least one radical chosen from (C₁-C₆)alkyl, (C₁-C₆)alkylcarbonyl, amido, and (C₁-C₆)alkylsulphonyl radicals; a carboxy(C₁-C₆)alkyl radical; a carbamyl(C₁-C₆)alkyl radical; a C₁-C₆ trifluoroalkyl radical; a tri(C₁-C₆)alkylsilane(C₁-C₆)alkyl radical; a C₁-C₆ sulphonamidoalkyl radical; a (C₁-C₆)alkylcarboxy(C₁-C₆)alkyl radical; a (C₁-C₆)alkylsulphinyl(C₁-C₆)alkyl radical; a (C₁-C₆)alkylsulphonyl(C₁-C₆)alkyl radical; a (C₁-C₆)alkylcarbonyl(C₁-C₆)alkyl radical; a N—(C₁-C₆)alkylcarbamyl(C₁-C₆)alkyl radical; and a N—(C₁-C₆)alkylsulphonamido(C₁-C₆)alkyl radical,
E, G, J, L and M, which may be identical or different, are chosen from carbon and nitrogen atoms, and form a ring chosen from pyridine, pyrimidine, pyrazine, triazine and pyridazine rings,
d is an integer ranging from 3 to 5,
x is equal to 0 or 1,
when x is equal to 0, the linker arm D is attached to the nitrogen atom,
when x is equal to 1, the linker arm D is attached to one ring member chosen from E, G, J, L, and M, when E, G, J, L or M is chosen from a carbon atom, and
T is a counterion.

63. The composition according to claim 62, wherein the ring members E, G, J, L and M form, with the nitrogen of the ring, a ring chosen from pyridine, pyrimidine, pyridazine, and pyrazine rings.

64. The composition according to claim 59, wherein x is equal to 0 and R is chosen from a hydroxyl radical; a C₁-C₆ alkyl radical; a C₁-C₆ monohydroxyalkyl radical; a C₂-C₆ polyhydroxyalkyl radical; a C₁-C₆ alkoxy radical; a tri(C₁-C₆)alkylsilane(C₁-C₆)alkyl radical; an amido radical; a C₁-C₆ alkylcarbonyl radical; an amino radical; an amino radical mono- or disubstituted with at least one radical chosen from (C₁-C₆)alkyl, (C₁-C₆)alkylcarbonyl, amido, and (C₁-C₆)alkylsulphonyl radicals; a C₁-C₆ monohydroxyalkyl radical; and a C₂-C₆ polyhydroxyalkyl radical; wherein the radical R is borne by a carbon atom.

65. The composition according to claim 59, wherein when x is equal to 1,
R₁₉ is chosen from a C₁-C₆ alkyl radical; a C₁-C₆ monohydroxyalkyl radical; a C₂-C₆ polyhydroxyalkyl radical; a C₁-C₆ aminoalkyl radical, wherein the amine is mono- or disubstituted with at least one radical chosen from (C₁-C₆)alkyl, (C₁-C₆)alkylcarbonyl, amido, and (C₁-C₆)alkylsulphonyl radicals; a C₁-C₆ carbamylalkyl radical; a tri(C₁-C₆)-alkylsilane(C₁-C₆)alkyl radical; a (C₁-C₆)alkylcarbonyl(C₁-C₆)alkyl radical; and a N—(C₁-C₆)alkylcarbamyl(C₁-C₆)alkyl radical; and
R is chosen from a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri(C₁-C₆)alkylsilane(C₁-C₆)alkyl radical, an amido radical, a C₁-C₆ alkylcarbonyl radical, an amino radical, and an amino radical mono- or disubstituted with at least one radical chosen from (C₁-C₆)alkyl, (C₁-C₆)alkylcarbonyl, amido, and (C₁-C₆)alkylsulphonyl radicals.

66. The composition according to claim 59, wherein R and R₁₉ are C₁-C₄ alkyl radicals which are optionally substituted.

67. The composition according to claim 47, wherein W₁ is chosen from 5-aminopyrazole, 5-hydroxypyrazole, pyrazolo[1,5-b]pyridine, pyrazolo[1,5-a]pyrimidine, pyrazolo[3,2-c]triazole, pyrazolo[1,5-b]triazole, aminopyrimidine, triaminopyrimidine, hydroxyaminopyrimidine, 2-aminopyridine, indoline, and indole radicals.

68. The composition according to claim 67, wherein W₁ is chosen from the 5-aminopyrazole and 5-hydroxypyrazole radicals of formula (R1).

69. The composition according to claim 68, wherein W₁ is chosen from 5-aminopyrazole and 5-hydroxypyrazole radicals wherein R₆ and R₁₁, which may be identical or different, are chosen from hydrogen atoms and linear and branched, saturated and unsaturated C₁-C₁₀ hydrocarbon-based chains, which optionally form at least one 4- to 8-membered carbon-based ring, wherein at least one carbon atom of the carbon-based chain, independently of each other, is optionally replaced with an entity chosen from oxygen, nitrogen and sulphur atoms, and SO₂ groups, and optionally substituted with at least one entity chosen from a halogen atom, and hydroxyl, amino, carboxyl, sulphonic, and thiol radicals; with the proviso that the radicals R₆ to R₁₂ do not comprise a peroxide bond, or a diazo or nitroso radical and the radical R₁₁ is not linked directly to the nitrogen atom via an oxygen, sulphur or nitrogen atom.

70. The composition according to claim 69, wherein R₆ and R₁₁, which are independent of each other, are chosen from hydrogen atoms and linear and branched, saturated and unsaturated C₁-C₄ hydrocarbon-based chains, which optionally form at least one 5- or 6-membered carbon-based ring, wherein the carbon atoms of the carbon-based chain, independently of each other, are optionally substituted with at least one entity chosen from a halogen atom, a hydroxyl radical, and amino radicals.

71. The composition according to claim 47, wherein W₁ is chosen from a compound of formula RIII 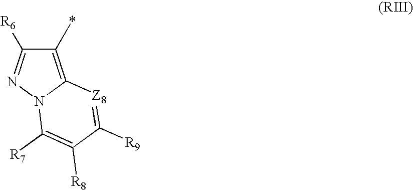
wherein R₆, R₇, R₈, and R₉, which are independent of each other, are chosen from:
hydrogen atoms,
linear and branched, saturated and unsaturated C₁-C₁₀ hydrocarbon-based chains, which optionally form at least one 4- to 8-membered carbon-based ring, wherein at least one carbon atom of the carbon-based chain, independently of the other carbon atoms, is optionally replaced with an entity chosen from oxygen, nitrogen and sulphur atoms, and SO₂ groups, and optionally substituted with at least one entity chosen from halogen atoms and hydroxyl, amino, carboxyl, sulphonic, and thiol radicals; with the proviso that the radicals R₆ to R₉ do not comprise a peroxide bond or diazo or nitroso radicals, and
Z₈ is chosen from a nitrogen atom and a CR₁₅ radical.

72. The composition according to claim 71, wherein W₁ is a pyrazolo[1,5-b]pyridine radical wherein R₆, R₇, R₈, R₉ and R₁₅, which may be identical or different, are chosen from
hydrogen atoms,
linear and branched, saturated and unsaturated C₁-C₁₀ hydrocarbon-based chains, which optionally form at least one 4- to 8-membered carbon-based ring, wherein at least one carbon atom of the carbon-based chain, independently of the other carbon atoms, is optionally replaced with an entity chosen from oxygen, nitrogen and sulphur atoms, and SO₂ groups, and optionally substituted with at least one entity chosen from halogen atoms and hydroxyl, amino, carboxyl, sulphonic, and thiol radicals; with the proviso that the radicals do not comprise a peroxide bond or diazo or nitroso radicals,
hydroxyl and amino radicals, the amine radical optionally substituted with a linear or branched, saturated or unsaturated C₁-C₄ hydrocarbon-based chain, which optionally forms at least one 5- or 6-membered carbon-based ring, wherein the carbon atoms of the carbon-based chain, independently of each other, are optionally substituted with at least one entity chosen from halogen atoms and hydroxyl and amino radicals.

73. The composition according to claim 72, wherein W₁ is a pyrazolo[1,5-b]pyridine radical wherein R₆, R₇, R₈, R₉ and R₁₅, which may be identical or different, are chosen from:
hydrogen atoms,
linear and branched, saturated and unsaturated C₁-C₁₀ hydrocarbon-based chains, which optionally form at least one 4- to 8-membered carbon-based ring, wherein at least one carbon atom of the carbon-based chain, independently of the other carbon atoms, is optionally replaced with an entity chosen from oxygen, nitrogen and sulphur atoms, and SO₂ groups, and optionally substituted with at least one entity chosen from halogen atoms and hydroxyl, amino, carboxyl, sulphonic and thiol radicals; with the proviso that the radicals do not comprise a peroxide bond or diazo or nitroso radicals,
hydroxyl or amino radicals, the amine radical is optionally substituted with a linear or branched, saturated or unsaturated C₁-C₄ hydrocarbon-based chain, which optionally forms at least one 6-membered carbon-based ring, wherein the carbon atoms of the carbon-based chain, which are independent of each other, optionally are substituted with at least one entity chosen from halogen atoms, and hydroxyl and amino radicals.

74. The composition according to claim 71, wherein W₁ is a pyrazolo[1,5-b]pyridine radical wherein R₆, R₇, R₈, R₉ and R₁₅, which may be identical or different, are chosen from:
hydrogen atoms,
linear and branched, saturated and unsaturated C₁-C₁₀ hydrocarbon-based chains, which optionally form at least one 4- to 8-membered carbon-based ring, wherein the carbon atoms of the carbon-based chain, independently of each other, optionally are substituted with at least one entity chosen from halogen atoms and hydroxyl, amino, monosubstituted or disubstituted amino, C₁-C₄ alkoxy, C₁-C₄ thioether, carboxyl, sulphonic, and thiol radicals;
hydroxyl and amino radicals, the amine optionally substituted with a linear or branched, saturated or unsaturated C₁-C₄ hydrocarbon-based chain, which optionally forms at least one 5- or 6-membered carbon-based ring, wherein the carbon atoms of the carbon-based chain, independently of each other, optionally are substituted with at least one entity chosen from halogen atoms and hydroxyl, and amino radicals.

75. The composition according to claim 72, wherein the radicals R₆, R₇, R₈, R₉ and R₁₅ are chosen from hydrogen atoms and linear and branched C₁-C₄ hydrocarbon-based chains which are optionally saturated or unsaturated, wherein the carbon atoms of the carbon-based chain, independently of each other, optionally are substituted with at least one entity chosen from halogen atoms, hydroxyl and amino radicals.

76. The composition according to claim 71, wherein W₁ is a pyrazolo[1,5-a]pyrimidine radical wherein
R₇ and R₉ are chosen from hydrogen atoms; linear and branched C₁-C₆ alkyl radicals; C₁-C₆ monohydroxyalkyl radicals; C₂-C₆ polyhydroxyalkyl radicals; C₁-C₆ aminoalkyl radicals and C₁-C₆ aminoalkyl radicals wherein the amine is mono- or disubstituted with at least one radical chosen from a (C₁-C₆)alkyl radical; a (C₁-C₆)alkylcarbonyl radical; a hydroxyl radical; and an amino radical, wherein the amino is optionally substituted with a linear or branched C₁-C₁₀ hydrocarbon-based chain, which optionally forms at least one 5- or 6-membered carbon-based ring which is saturated or unsaturated, wherein the carbon atoms of the carbon-based chain, independently of each other, optionally are substituted with at least one entity chosen from halogen atoms and hydroxyl and amino radicals;
R₆ and R₈ are chosen from hydrogen atoms; linear and branched C₁-C₆ alkyl radicals; C₁-C₆ monohydroxyalkyl radicals; C₂-C₆ polyhydroxyalkyl radicals; C₁-C₆ aminoalkyl radicals; and C₁-C₆ aminoalkyl radicals wherein the amine is mono- or disubstituted with at least one radical chosen from (C₁-C₆)alkyl and (C₁-C₆)alkylcarbonyl radicals.

77. The composition according to claim 76, wherein
R₇ and R₉ are chosen from hydrogen atoms; linear and branched C₁-C₄ alkyl radicals; C₁-C₄ monohydroxyalkyl radicals; C₂-C₄ polyhydroxyalkyl radicals; C₁-C₄ aminoalkyl radicals; and C₁-C₄ aminoalkyl radicals wherein the amine is mono- or disubstituted with at least one radical chosen from (C₁-C₂)alkyl, hydroxyl, and amino radicals, wherein the amino is optionally substituted with a linear or branched C₁-C₄ hydrocarbon-based chain, wherein the carbon atoms of the carbon-based chain, independently of each other, optionally are substituted with at least one entity chosen from hydroxyl and amino radicals, and
R₆ and R₈ are chosen from hydrogen atoms; linear and branched C₁-C₄ alkyl radicals; C₁-C₄ monohydroxyalkyl radicals; C₂-C₄ polyhydroxyalkyl radicals; C₁-C₄ aminoalkyl radicals; C₁-C₄ aminoalkyl radicals wherein the amine is mono- or disubstituted with at least one radical chosen from (C₁-C₂)alkyl and C₁-C₂ alkoxy radicals.

78. The composition according to claim 77, wherein R₆, R₇, R₈ and R₉ are chosen from hydrogen atoms; C₁-C₄ alkyl radicals; amino radicals; C₁-C₄ mono- and dialkylamino radicals; C₁-C₄ hydroxyalkyl radicals; and C₁-C₂ alkoxy radicals.

79. The composition according to claim 47, wherein the compound of formula (I) is a cationic compound substituted with at least one onium radical Z.

80. The composition according to claim 79, wherein at least one of the radicals R₁ and R₂ is an onium radical Z.

81. The composition according to claim 80, wherein R₁ and R₂ form a ring of formula (II) wherein R′ is an onium radical Z.

82. The composition according to claim 81, wherein Y is NR′₆R′₇.

83. The composition according to claim 47, wherein the compound of formula (I) is a compound of the following formula 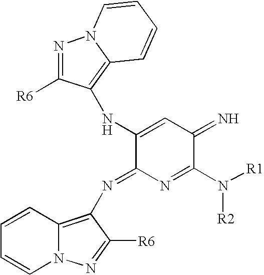
wherein R₁ and R₂, which are independent of each other, are chosen from:
hydrogen atoms,
linear and branched, saturated and unsaturated C₁-C₁₀ hydrocarbon-based chains, which optionally form at least one 4- to 8-membered carbon-based ring, wherein at least one carbon atom of the carbon-based chain, independently from the other carbon atoms, optionally is replaced with an entity chosen from oxygen, nitrogen and sulphur atoms, and SO₂ groups, and optionally substituted with at least one entity chosen from halogen atoms and hydroxyl, amino, C₁-C₂ (di)alkylamino, C₁-C₂ alkoxy, carboxyl, sulphonic, and thiol radicals; with the proviso that R₁ and R₂ do not comprise a peroxide bond, or a diazo or nitroso radical, and R₁ and R₂ are not directly linked to the nitrogen atom via an oxygen, sulphur or nitrogen atom, or SO₂ group, and
an onium radical Z, or alternatively
R₁ and R₂ form, with the nitrogen atom to which they are attached, a ring of formula (II): 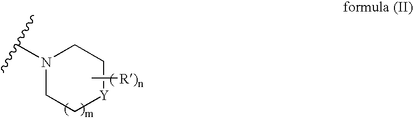
wherein
R′ is chosen from:
a hydrogen atom;
a halogen atom;
a C₁-C₄ alkyl radical optionally substituted with at least one radical chosen from hydroxyl, carboxyl, C₁-C₄ alkoxycarbonyl, (C₁-C₄)alkylamido((C₁-C₄)alkylCONH—), (C₁-C₄)alkylcarbamoyl ((C₁-C₄)alkylNHCO—), (C₁-C₄)alkylsulphonyl ((C₁-C₄)alkylSO₂—), C₁-C₄ alkoxy, (C₁-C₄)alkylsulphonamido ((C₁-C₄)alkylSO₂NH—), (C₁-C₄)alkylsulphamoyl ((C₁-C₄)alkylNHSO₂—), and onium Z radicals;
a NR′₃R′₄ radical;
a carboxyl radical;
a C₁-C₄ alkoxycarbonyl radical;
a (C₁-C₄)alkylamido radical ((C₁-C₄)alkylCONH—);
a (C₁-C₄)alkylsulphonyl radical (alkylSO₂—);
an alkylsulphonamido radical ((C₁-C₄)alkylSO₂NH—);
a hydroxyl radical;
a C₁-C₄ alkoxy radical;
a C₂-C₄ hydroxyalkoxy radical;
a (C₁-C₄)alkylcarbamoyl radical ((C₁-C₄)alkylNHCO—);
(C₁-C₄)alkylsulphamoyl ((C₁-C₄)alkyl-NH—SO₂—);
a C₁-C₄ thioether radical;
a sulphonic radical (SO₃H) and the addition salts thereof; and
an onium radical Z,
wherein R′₃ and R′₄, which may be identical or different, are chosen from hydrogen atom; and C₁-C₄ alkyl radicals optionally substituted with at least one radical chosen from hydroxyl, C₁-C₄ alkoxy, amino, mono- and dialkylamino, (C₁-C₄)alkylCO—, (C₁-C₄)alkylNHCO—, and (C₁-C₄)alkylSO₂— radicals,
n is an integer ranging from 1 to 8,
m is an integer ranging from 0 to 3, and
Y is chosen from a oxygen atom, a CR′ radical, a NR′₅ radical, and a NR′₆R′₇ radical wherein
R′₅ is chosen from a hydrogen atom and a linear or branched, saturated or unsaturated C₁-C₁₀ hydrocarbon-based chain, wherein at least one carbon atom of the carbon-based chain, independently of the other carbon atoms, optionally is replaced with an entity chosen from oxygen, nitrogen and sulphur atoms, and SO₂ groups, and optionally substituted with at least one entity chosen from halogen atoms and hydroxyl, amino, carboxyl, sulphonic, and thiol radicals; with the proviso that R′₅ does not comprise a peroxide bond, or a diazo or nitroso radical, and R′₅ is not directly linked to the nitrogen atom via an oxygen, sulphur or nitrogen atom,
R′₆ and R′₇, which are independent of each other, are chosen from linear and branched C₁-C₁₀ hydrocarbon-based chains, which are saturated or unsaturated, wherein at least one carbon atom of the carbon-based chain, independently of the other carbon atoms, optionally is replaced with an entity chosen from oxygen, nitrogen and sulphur atoms, and SO₂ groups, and optionally substituted with at least one entity chosen from halogen atoms and hydroxyl, amino, carboxyl, sulphonic, and thiol radicals; with the proviso that R′₆ and R′₇ do not comprise a peroxide bond, or a diazo or nitroso radical, and R′₆ and R′₇ are not directly linked to the nitrogen atom via an oxygen, sulphur or nitrogen atom,
R₆ is chosen from:
a hydrogen atom,
a linear or branched, saturated or unsaturated C₁-C₁₀ hydrocarbon-based chain, which optionally forms at least one 4- to 8-membered carbon-based ring, wherein at least one carbon atom of the carbon-based chain, which may be identical or different, optionally is replaced with an entity chosen from oxygen, nitrogen and sulphur atoms, and SO₂ groups, and optionally is substituted with at least one entity chosen from halogen atoms and hydroxyl, amnio, carboxyl, sulphonic, and thiol radicals; with the proviso that the radical R₆ does not comprise a peroxide bond or diazo or nitroso radicals.

84. The composition according to claim 47, wherein the compound of formula (I) is chosen from

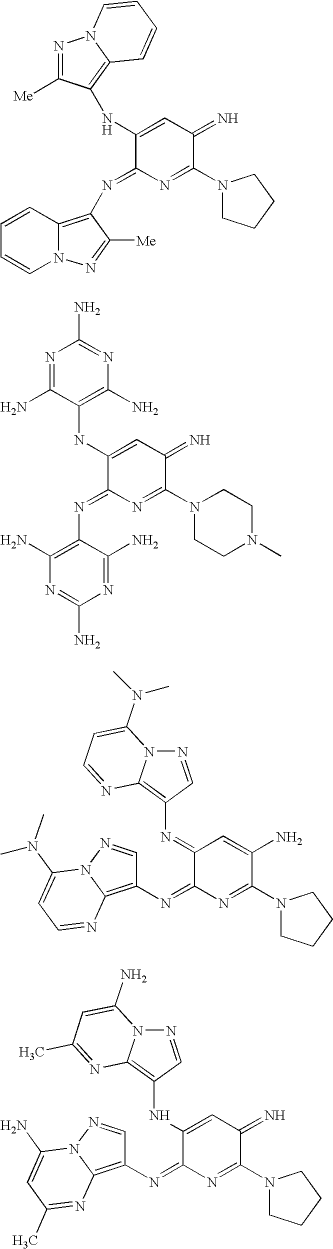

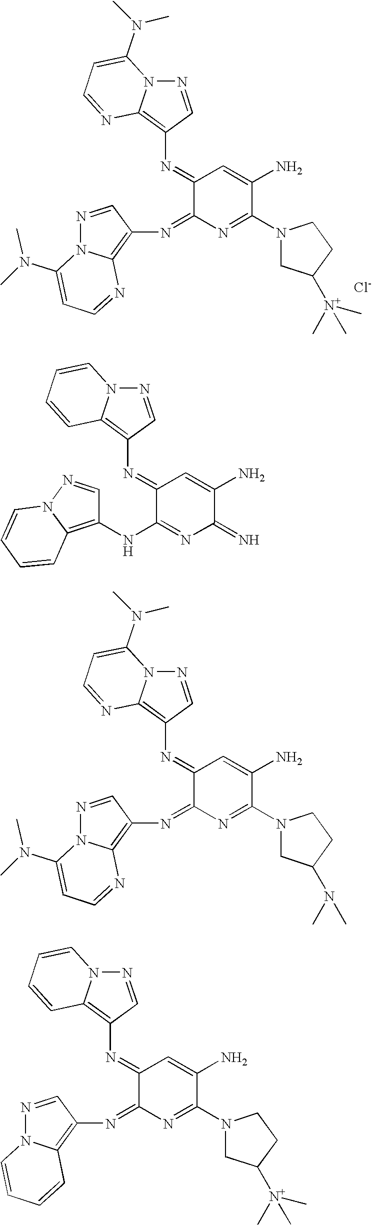

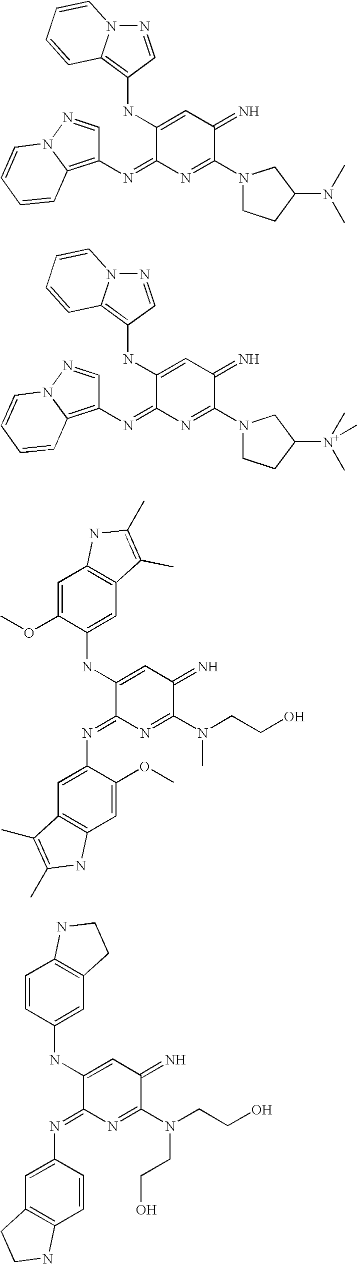

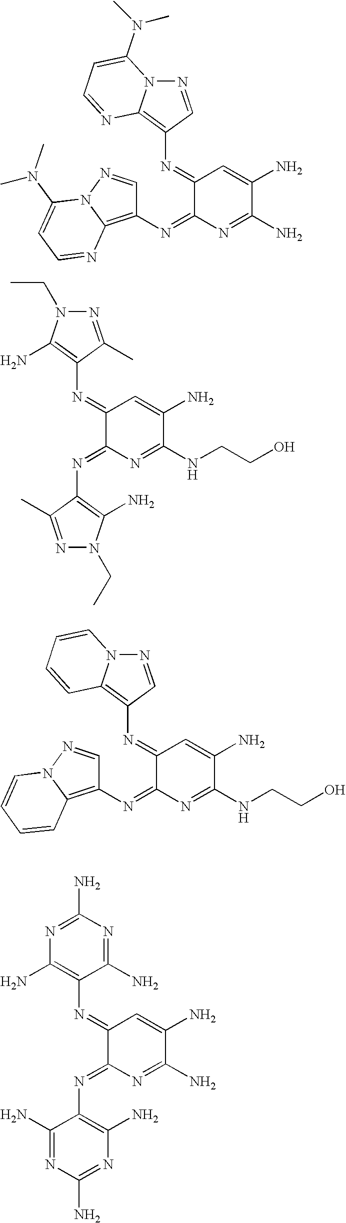

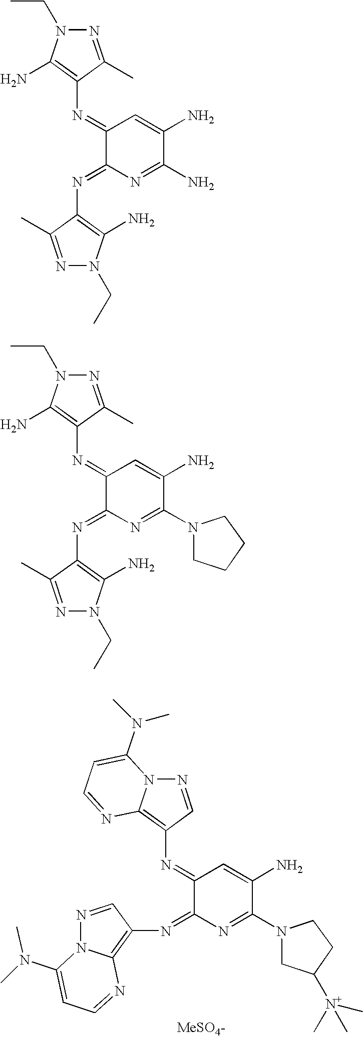

85. The composition according to claim 47, wherein the compound of formula (I) is present in an amount ranging from 0.01% to 10% by weight, relative to the total weight of the composition.

86. The composition according to claim 47, further comprising at least one oxidation base chosen from para-phenylenediamines, bis(phenyl)alkylenediamines, para-aminophenols, ortho-aminophenols, heterocyclic bases, and the acid-addition salts thereof.

87. The composition according to claim 86, wherein the at least one oxidation base is present in an amount ranging from 0.001% to 10% by weight, relative to the total weight of the composition.

88. The composition according to claim 47, further comprising at least one coupler chosen from meta-phenylenediamines, meta-aminophenols, meta-diphenols, naphthalene-based couplers, heterocyclic couplers, and the acid-addition salts thereof.

89. The composition according to claim 47, further comprising at least one oxidizing agent.

90. A direct dye of formula (I) comprising, in a suitable medium, at least one compound of formula (I), or an addition salt thereof: 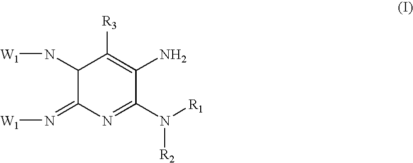
wherein
R₁ and R₂, which are independent of each other, are chosen from:
hydrogen atoms,
linear and branched, saturated and unsaturated C₁-C₁₀ hydrocarbon-based chains, which optionally form at least one 4- to 8-membered carbon-based ring, wherein at least one carbon atom of the carbon-based chain, independently of the other carbon atoms, is optionally replaced with an entity chosen from oxygen, nitrogen and sulphur atoms and SO₂ groups, and optionally substituted with at least one entity chosen from halogen atoms and hydroxyl, amino, C₁-C₂ (di)alkylamino, C₁-C₂ alkoxy, carboxyl, sulphonic, and thiol radicals; with the proviso that R₁ and R₂ do not comprise a peroxide bond or diazo or nitroso radical, and R₁ and R₂ are not directly linked to the nitrogen atom via an oxygen, sulphur or nitrogen atom or an SO₂ group, and
onium radical Z, or alternatively
R₁ and R₂ form, with the nitrogen atom to which they are attached, a ring of formula (II): 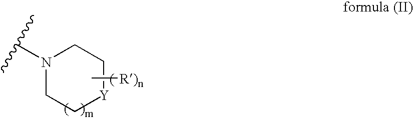
wherein
R′ is chosen from:
a hydrogen atom;
a halogen atom;
a C₁-C₄ alkyl radical optionally substituted with at least one radical chosen from hydroxyl, carboxyl, C₁-C₄ alkoxycarbonyl, (C₁-C₄)alkylamido((C₁-C₄)alkylCONH—), (C₁-C₄)alkylcarbamoyl ((C₁-C₄)alkylNHCO—), (C₁-C₄)alkylsulphonyl ((C₁-C₄)alkylSO₂—), C₁-C₄ alkoxy, (C₁-C₄)alkylsulphonamido ((C₁-C₄)alkylSO₂NH—), (C₁-C₄)alkylsulphamoyl ((C₁-C₄)alkylNHSO₂—), and onium Z radicals;
a NR′₃R′₄ radical;
a carboxyl radical;
a C₁-C₄ alkoxycarbonyl radical;
a (C₁-C₄)alkylamido radical ((C₁-C₄)alkylCONH—);
a (C₁-C₄)alkylsulphonyl radical (alkylSO₂—);
an alkylsulphonamido radical ((C₁-C₄)alkylSO₂NH—);
a hydroxyl radical;
a C₁-C₄ alkoxy radical;
a C₂-C₄ hydroxyalkoxy radical;
a (C₁-C₄)alkylcarbamoyl radical ((C₁-C₄)alkylNHCO—);
(C₁-C₄)alkylsulphamoyl ((C₁-C₄)alkyl-NH—SO₂—);
a C₁-C₄ thioether radical;
a sulphonic radical (SO₃H) and the addition salts thereof; and
an onium radical Z,
wherein R′₃ and R′₄, which may be identical or different, are chosen from hydrogen atoms and C₁-C₄ alkyl radicals optionally substituted with at least one radical chosen from hydroxyl, C₁-C₄ alkoxy, amino, mono- and dialkylamino, (C₁-C₄)alkylCO—, (C₁-C₄)alkylNHCO—, and (C₁-C₄)alkylSO₂— radicals,
n is an integer ranging from 1 to 8,
m is an integer ranging from 0 to 3, and
Y is chosen from a oxygen atom, a CR′ radical, a NR′₅ radical, and a NR′₆R′₇ radical wherein
R′₅ is chosen from a hydrogen atom and linear and branched, saturated and unsaturated C₁-C₁₀ hydrocarbon-based chains, wherein at least one carbon atom of the carbon-based chain, independently of the other carbon atoms, is optionally replaced with an entity chosen from oxygen, nitrogen and sulphur atoms, and SO₂ groups, and optionally substituted with at least one entity chosen from halogen atoms and hydroxyl, amino, carboxyl, sulphonic, and thiol radicals; with the proviso that R′₅ does not comprise a peroxide bond, or diazo or nitroso radicals, and R′₅ is not directly linked to the nitrogen atom via an oxygen, sulphur or nitrogen atom,
R′₆ and R′₇, which are independent of each other, are chosen from linear and branched C₁-C₁₀ hydrocarbon-based chains, which are saturated or unsaturated, wherein at least one carbon atom of the carbon-based chain, independently of each other, may optionally be replaced with an entity chosen from oxygen, nitrogen and sulphur atoms, and SO₂ groups, and optionally substituted with at least one entity chosen from halogen atoms and hydroxyl, amino, carboxyl, sulphonic, and thiol radicals; with the proviso that R′₆ and R′₇ do not comprise a peroxide bond, or diazo or nitroso radical, and R′₆ and R′₇ are not directly linked to the nitrogen atom via an oxygen, sulphur or nitrogen atom,
R₃ is chosen from:
a hydrogen atom,
linear and branched, saturated and unsaturated C₁-C₁₀ hydrocarbon-based chains, which optionally form at least one 4- to 8-membered carbon-based ring, wherein at least one carbon atom of the carbon-based chain, independently of the other carbon atoms, is optionally replaced with an entity chosen from oxygen, nitrogen and sulphur atoms, and SO₂ groups, and optionally substituted with at least one entity chosen from halogen atoms and hydroxyl, amino, C₁-C₂ (di)alkylamino, C₁-C₂ alkoxy, carboxyl, sulphonic, and thiol radicals; with the proviso that R₃ does not comprise a peroxide bond, or diazo or nitroso radical,
a NR′₁R′₂ radical, wherein R′₁ and R′₂ are defined as R₁ and R₂,
W₁ is chosen from an aromatic heterocyclic radical chosen from the following radicals: 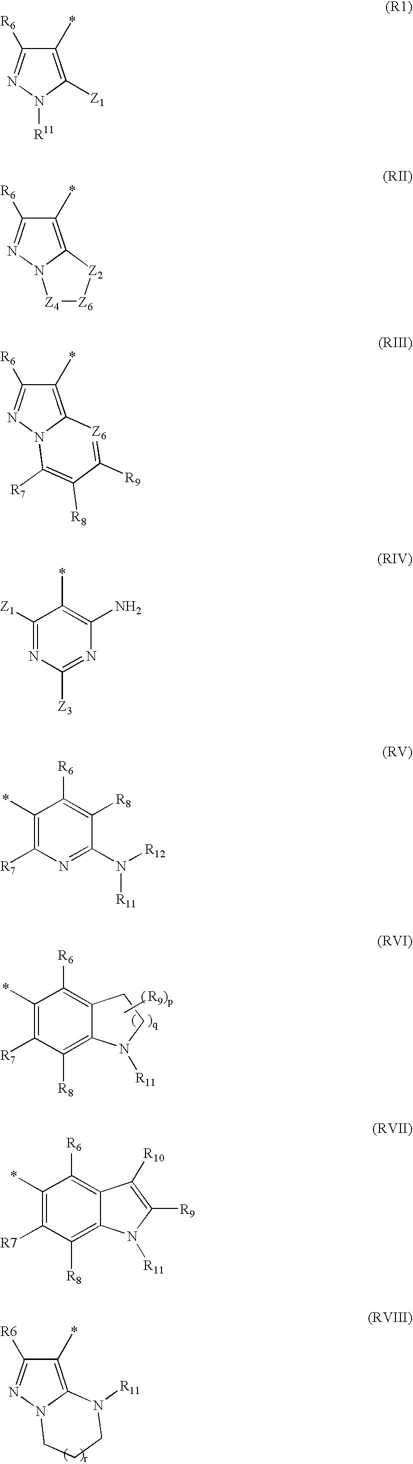
Z₁ and Z₃, which are independent of each other, are chosen from hydrogen atoms, hydroxyl radicals and NR₁₁R₁₂ radicals,
Z₂, Z₄ and Z₆, which are independent of each other, are chosen from nitrogen atoms, CR₁₂ radicals, and NR₁₁ radicals, wherein at least one of Z₂, Z₄, and Z₆ is a CR₁₂ radical and wherein there are no more than three contiguous nitrogen atoms,
Z₈ is chosen from a nitrogen atom and a CR₁₅ radical,
R₆, R₇, R₈, R₉, R₁₀, R₁₁, R₁₂ and R₁₅, which are independent of each other, are chosen from:
hydrogen atoms,
linear and branched, saturated and unsaturated C₁-C₁₀ hydrocarbon-based chains, which optionally form at least one 4- to 8-membered carbon-based ring, wherein at least one carbon atom of the carbon-based chain, independently of the other carbon atoms, is optionally replaced with an entity chosen from oxygen, nitrogen and sulphur atoms, and SO₂ groups, and optionally substituted with at least one entity chosen from halogen atoms and hydroxyl, amnio, carboxyl, sulphonic, and thiol radicals; with the proviso that the radicals R₆ to R₁₂ and R₁₅ do not comprise a peroxide bond, or diazo or nitroso radical, and the radical R₁₁ is not directly linked to the nitrogen atom via an oxygen, sulphur or nitrogen atom,
p ranges from 4 to 8,
q ranges from 1 to 3, and
r ranges from 0 to 2,
* indicates a point of attachment of W₁ in formula (I).

91. A process for dyeing keratin fibers comprising applying to said fibers for a period that is sufficient to obtain a desired coloration a dye composition comprising, in a suitable medium, at least one compound of formula (I), or an addition salt thereof: 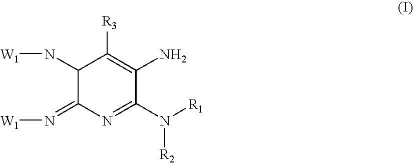
wherein
R₁ and R₂, which are independent of each other, are chosen from:
hydrogen atoms,
linear and branched, saturated and unsaturated C₁-C₁₀ hydrocarbon-based chains, which optionally form at least one 4- to 8-membered carbon-based ring, wherein at least one carbon atom of the carbon-based chain, independently of the other carbon atoms, is optionally replaced with an entity chosen from oxygen, nitrogen and sulphur atoms and SO₂ groups, and optionally substituted with at least one entity chosen from halogen atoms and hydroxyl, amino, C₁-C₂ (di)alkylamino, C₁-C₂ alkoxy, carboxyl, sulphonic, and thiol radicals; with the proviso that R₁ and R₂ do not comprise a peroxide bond, or diazo or nitroso radical, and R₁ and R₂ are not directly linked to the nitrogen atom via an oxygen, sulphur or nitrogen atom or an SO₂ group, and
an onium radical Z, or alternatively
R₁ and R₂ form, with the nitrogen atom to which they are attached, a ring of formula (II): 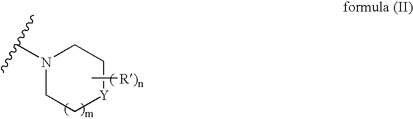
wherein
R′ is chosen from:
a hydrogen atom;
a halogen atom;
a C₁-C₄ alkyl radical optionally substituted with at least one radical chosen from hydroxyl, carboxyl, C₁-C₄ alkoxycarbonyl, (C₁-C₄)alkylamido((C₁-C₄)alkylCONH—), (C₁-C₄)alkylcarbamoyl ((C₁-C₄)alkylNHCO—), (C₁-C₄)alkylsulphonyl ((C₁-C₄)alkylSO₂—), C₁-C₄ alkoxy, (C₁-C₄)alkylsulphonamido ((C₁-C₄)alkylSO₂NH—), (C₁-C₄)alkylsulphamoyl ((C₁-C₄)alkylNHSO₂—), and onium Z radicals;
a NR′₃R′₄ radical;.
a carboxyl radical;
a C₁-C₄ alkoxycarbonyl radical;
a (C₁-C₄)alkylamido radical ((C₁-C₄)alkylCONH—);
a (C₁-C₄)alkylsulphonyl radical (alkylSO₂—);
an alkylsulphonamido radical ((C₁-C₄)alkylSO₂NH—);
a hydroxyl radical;
a C₁-C₄ alkoxy radical;
a C₂-C₄ hydroxyalkoxy radical;
a (C₁-C₄)alkylcarbamoyl radical ((C₁-C₄)alkylNHCO—);
(C₁-C₄)alkylsulphamoyl ((C₁-C₄)alkyl-NH—SO₂—);
a C₁-C₄ thioether radical;
a sulphonic radical (SO₃H) and the addition salts thereof; and
an onium radical Z,
wherein R′₃ and R′₄, which may be identical or different, are chosen from hydrogen atoms and C₁-C₄ alkyl radicals optionally substituted with at least one radical chosen from hydroxyl, C₁-C₄ alkoxy, amino, mono- and dialkylamino, (C₁-C₄)alkylCO—, (C₁-C₄)alkylNHCO—, and (C₁-C₄)alkylSO₂— radicals,
n is an integer ranging from 1 to 8,
m is an integer ranging from 0 to 3, and
Y is chosen from a oxygen atom, a CR′ radical, a NR′₅ radical, and a NR′₆R′₇ radical wherein
R′₅ is chosen from a hydrogen atom and linear and branched, saturated and unsaturated C₁-C₁₀ hydrocarbon-based chains, wherein at least one carbon atom of the carbon-based chain, independently of the other carbon atoms, is optionally replaced with an entity chosen from oxygen, nitrogen and sulphur atoms, and SO₂ groups, and optionally substituted with at least one entity chosen from halogen atoms and hydroxyl, amino, carboxyl, sulphonic, and thiol radicals; with the proviso that R′₅ does not comprise a peroxide bond, or diazo or nitroso radical, and R′₅ is not directly linked to the nitrogen atom via an oxygen, sulphur or nitrogen atom,
R′₆ and R′₇, which are independent of each other, are chosen from linear and branched, saturated and unsaturated C₁-C₁₀ hydrocarbon-based chains, wherein at least one carbon atom of the carbon-based chain, independently of the other carbon atoms, optionally is replaced with an entity chosen from oxygen, nitrogen and sulphur atoms, and SO₂ groups, and optionally substituted with at least one halogen atom, hydroxyl, amino, carboxyl, sulphonic, and thiol radicals; with the proviso that R′₆ and R′₇ do not comprise a peroxide bond or diazo or nitroso radicals, and R′₆ and R′₇ are not directly linked to the nitrogen atom via an oxygen, sulphur or nitrogen atom,
R₃ is chosen from:
a hydrogen atom,
a linear and branched, saturated and unsaturated C₁-C₁₀ hydrocarbon-based chains, which optionally form at least one 4- to 8-membered carbon-based ring, wherein at least one carbon atom of the carbon-based chain, independently of the other carbon atoms, is optionally replaced with an entity chosen from oxygen, nitrogen and sulphur atoms, and SO₂ groups, and optionally substituted with at least one entity chosen from halogen atoms and hydroxyl, amino, C₁-C₂ (di)alkylamino, C₁-C₂ alkoxy, carboxyl, sulphonic, and thiol radicals; with the proviso that R₃ does not comprise an entity chosen from a peroxide bond, or diazo or nitroso radical,
a NR′₁R′₂ radical, wherein R′₁ and R′₂ being defined as R₁ and R₂,
W₁ is chosen from an aromatic heterocyclic radical chosen from the following radicals: 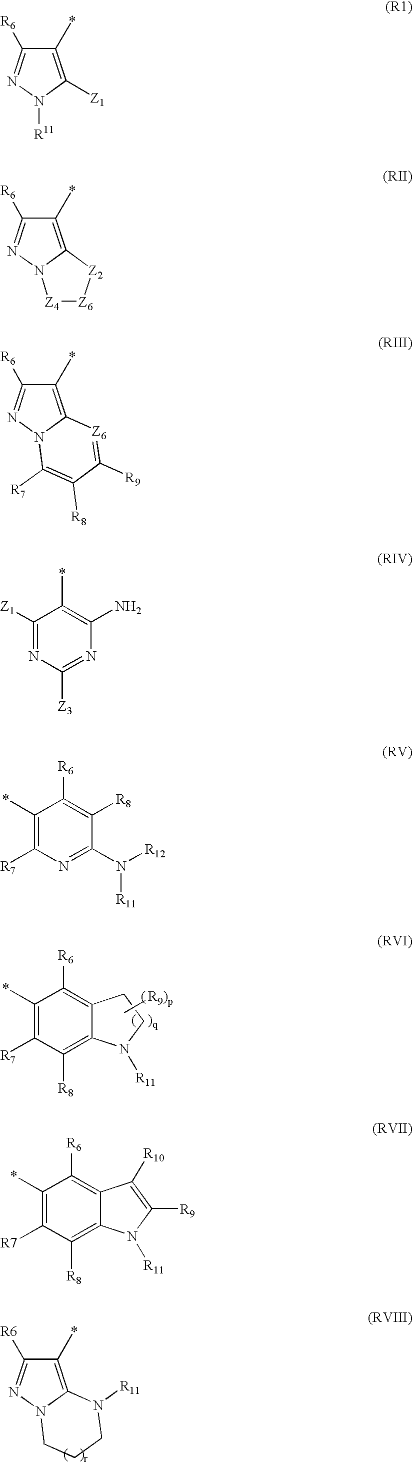
Z₁ and Z₃, which are independent of each other, are chosen from hydrogen atoms, hydroxyl radicals and NR₁₁R₁₂ radicals,
Z₂, Z₄ and Z₆, which are independent of each other, are chosen from nitrogen atoms, CR₁₂ radicals, and NR₁₁ radicals, wherein at least one of Z₂, Z₄, and Z₆ is chosen from a CR₁₂ radical and wherein there are no more than three contiguous nitrogen atoms,
Z₈ is chosen from a nitrogen atom and a CR₁₅ radical,
R₆, R₇, R₈, R₉, R₁₀, R₁₁, R₁₂ and R₁₅, which are independent of each other, are chosen from:
hydrogen atoms,
linear and branched, saturated and unsaturated C₁-C₁₀ hydrocarbon-based chains, which optionally form at least one 4- to 8-membered carbon-based ring, wherein at least one carbon atom of the carbon-based chain, independently of the other carbon atoms, is optionally replaced with an entity chosen from oxygen, nitrogen and sulphur atoms, and SO₂ groups, and optionally is substituted with at least one entity chosen from halogen atoms and hydroxyl, amnio, carboxyl, sulphonic, and thiol radicals; with the proviso that the radicals R₆ to R₁₂ and R₁₅ do not comprise a peroxide bond, or diazo or nitroso radical, and the radical R₁₁ is not directly linked to the nitrogen atom via an oxygen, sulphur or nitrogen atom,
p ranges from 4 to 8,
q ranges from 1 to 3, and
r ranges from 0 to 2,
* indicates a point of attachment of W₁ in formula (I).
